(19) United States
(12) Patent Application Publication
(10) Pub. No.: US 2025/0367083 A1
(43) Pub. Date: Dec. 4, 2025

(54) DEGRADABLE DELIVERY PARTICLES MADE FROM REDOX-INITIATOR-MODIFIED CHITOSAN

(71) Applicant: Encapsys, LLC, Appleton, WI (US)

(72) Inventors: Linsheng Feng, Menasha, WI (US); Travis Ian Bardsley, Appleton, WI (US); Mattia Collu, Saint-Gilles (BE); Johan Smets, Lubbeek (BE); Meagan Marie Kochel, Appleton, WI (US); Susana Fernandez-Prieto, Brussels (BE); Ariel Lebron, Cincinnati, OH (US); Sonia Marcela Malagon Gomez, Appleton, WI (US); Cedric Marc Tahon, Ghent (BE)

(73) Assignee: Encapsys, LLC, Appleton, WI (US)

(21) Appl. No.: 18/874,150

(22) PCT Filed: Nov. 29, 2023
(86) PCT No.: PCT/US2023/081498

§ 371 Date: Dec. 12, 2024

Related U.S. Application Data
(60) Provisional application No. 63/429,240, filed on Dec. 1, 2022.

(51) Int. Cl. A61K 8/02; A61Q 13/00; C11D 3/50; C11D 17/00

(52) U.S. Cl. CPC A61K 8/0279; A61Q 13/00; C11D 3/505; C11D 17/0039; A61K 2800/412; A61K 2800/654

(57) ABSTRACT

A population of core-shell delivery particles comprising a benefit agent core material and a shell encapsulating the core material is described, along with a process for forming such delivery particles and articles of manufacture. The shell is the reaction product of a crosslinking agent and a modified chitosan. Chitosan is treated with a mixture of an acid and redox initiator comprising a persulfate or peroxide, which results in an enhanced polymeric shell. The delivery particle of the invention has improved release characteristics, with enhanced degradation characteristics in OECD test method 301B.

### CROSS REFERENCE TO RELATED APPLICATIONS

### Statement of Joint Research

Encapsys, LLC and The Procter & Gamble Company executed a Joint Research Agreement on or about Jul. 29, 2021 and this invention was made as a result of activities undertaken within the scope of that Joint Research Agreement between the parties that was in effect on or before the date of this invention.

### FIELD OF THE INVENTION

This invention relates to capsule manufacturing processes and biodegradable delivery particles produced by such processes, the delivery particles containing a core material and a shell encapsulating the core, the shell comprising a reaction product of a cross-linking agent and polysaccharide.

### DESCRIPTION OF THE RELATED ART

Microencapsulation is a process where droplets of liquids, particles of solids or gasses are enclosed inside a solid shell and are generally in the micro-size range. The core material is separated from the surrounding environment by the shell. Microencapsulation technology has a wide range of commercial applications for different industries. Overall, capsules are capable of one or more of (i) providing stability of a formulation or material via the mechanical separation of incompatible components, (ii) protecting the core material from the surrounding environment, (iii) masking or hiding an undesirable attribute of an active ingredient and (iv) controlling or triggering the release of the active ingredient to a specific time or location. All of these attributes can lead to an increase of the shelf-life of several products and a stabilization of the active ingredient in liquid formulations.

Various processes for microencapsulation, and exemplary methods and materials are set forth in Schwantes (U.S. Pat. No. 6,592,990), Nagai et al. (U.S. Pat. No. 4,708,924), Baker et al. (U.S. Pat. No. 4,166,152), Wojciak (U.S. Pat. No. 4,093,556), Matsukawa et al. (U.S. Pat. No. 3,965,033), Ozono (U.S. Pat. No. 4,588,639), Irgarashi et al. (U.S. Pat. No. 4,610,927), Brown et al. (U.S. Pat. No. 4,552,811), Scher (U.S. Pat. No. 4,285,720), Jahns et al. (U.S. Pat. Nos. 5,596,051 and 5,292,835), Matson (U.S. Pat. No. 3,516,941), Foris et al. (U.S. Pat. Nos. 4,001,140; 4,087,376; 4,089,802 and 4,100,103), Greene et al. (U.S. Pat. Nos. 2,800,458; 2,800,457 and 2,730,456), Clark (U.S. Pat. No. 6,531,156), Hoshi et al. (U.S. Pat. No. 4,221,710), Hayford (U.S. Pat. No. 4,444,699), Hasler et al. (U.S. Pat. No. 5,105,823), Stevens (U.S. Pat. No. 4,197,346), Riecke (U.S. Pat. No. 4,622,267), Greiner et al. (U.S. Pat. No. 4,547,429), and Tice et al. (U.S. Pat. No. 5,407,609), among others and as taught by Herbig in the chapter entitled “Microencapsulation” in Kirk-Othmer Encyclopedia of Chemical Technology, V.16, pages 438-463.

Core-shell encapsulation is useful to preserve actives, such as benefit agents, in harsh environments and to release them at the desired time, which may be during or after use of goods incorporating the encapsulates. Among various mechanisms that can be used for release of benefit agent from the encapsulates, the one commonly relied upon is mechanical rupture of the capsule shell through friction or pressure. Selection of mechanical rupture as the release mechanism constitutes another challenge to the manufacturer, as rupture must occur at specific desired times, even if the capsules are subject to mechanical stress prior to the desired release time.

Industrial interest for encapsulation technology has led to the development of several polymeric capsules chemistries which attempt to meet the requirements of biodegradability, low shell permeability, high deposition, targeted mechanical properties and rupture profile. Increased environmental concerns have put the polymeric capsules under scrutiny, therefore manufacturers have started investigating sustainable solutions for the encapsulation of benefit agents.

Biodegradable materials exist and are able to form delivery particles via coacervation, spray-drying or phase inversion precipitation. However, the delivery particles formed using these materials and techniques are highly porous and not suitable for aqueous compositions containing surfactants or other carrier materials, since the benefit agent is prematurely released to the composition.

Non-leaky and performing delivery particles in aqueous surfactant-based compositions exist, however due to its chemical nature and cross-linking, they are not biodegradable.

Encapsulation can be found in areas as diverse as pharmaceuticals, personal care, textiles, food, coatings and agriculture. In addition, the main challenge faced in encapsulation is that a complete retention of the encapsulated active within the capsule is required throughout the whole supply chain, until a controlled or triggered release of the core material is applied. There are significantly limited microencapsulation technologies that can fulfill the rigorous criteria for long-term retention and active protection capability for commercial needs, especially when it comes to encapsulation of small molecules. A further challenge in certain applications and formulations is compatibility of the delivery particles with finished matrices such as laundry formulations or fabric care formulations. Stability within such matrices is enhanced with the compositions of the invention.

Delivery particles are needed that are biodegradable yet have high structural integrity so as to reduce leakage and resist damage from harsh environments. Moreover a need exists for degradable delivery particles having improved performance and which are compatible with end use formulations.

### Definitions

As used herein, reference to the term “(meth)acrylate” or “(meth)acrylic” is to be understood as referring to both the acrylate and the methacrylate versions of the specified monomer, oligomer and/or prepolymer, (for example “isobornyl (meth)acrylate” indicates that both isobornyl methacrylate and isobornyl acrylate are possible, similarly reference to alkyl esters of (meth)acrylic acid indicates that both alkyl esters of acrylic acid and alkyl esters of methacrylic acid are possible, similarly poly(meth)acrylate indicates that both polyacrylate and polymethacrylate are possible). Similarly, the use of the phrase “prepolymer” means that the referenced material may exist as a prepolymer or combination of oligomers and prepolymers. Similarly, it is to be understood that the general reference herein to (meth)acrylate or (meth)acrylates, e.g., “water soluble (meth)acrylates,” “water phase (meth)acrylate,” etc., is intended to cover or include the (meth)acrylate monomers and/or oligomers. Additionally, the descriptors “water soluble or dispersible,” water soluble,” and “water dispersible” when referencing certain (meth)acrylate monomers and/or oligomers or initiators means that the specified component is soluble or dispersible in the given matrix solution on its own or in the presence of a suitable solubilizer or emulsifier or upon attainment of certain temperatures and/or pH.

Each alkyl moiety herein, unless otherwise indicated, can be from C₁ to C₈, or even from C₁ to C₂₄. Poly(meth)acrylate materials are intended to encompass a broad spectrum of polymeric materials including, for example, polyester poly(meth)acrylates, urethane and polyurethane poly(meth)acrylates (especially those prepared by the reaction of a hydroxyalkyl (meth)acrylate with a polyisocyanate or a urethane polyisocyanate), methyl cyanoacrylate, ethyl cyanoacrylate, diethylene glycol di(meth)acrylate, trimethylolpropane tri(meth)acrylate, ethylene glycol di(meth)acrylate, allyl (meth)acrylate, glycidyl (meth)acrylate, (meth)acrylate functional silicones, di-, tri- and tetraethylene glycol di(meth)acrylate, dipropylene glycol di(meth)acrylate, polyethylene glycol di(meth)acrylate, di(pentamethylene glycol) di(meth)acrylate, ethylene di(meth)acrylate, neopentyl glycol di(meth)acrylate, ethoxylated bisphenol A di(meth)acrylates, bisphenol A di(meth)acrylates, diglycerol di(meth)acrylate, tetraethylene glycol dichloroacrylate, 1,3-butanediol di(meth)acrylate, neopentyl di(meth)acrylate, polyethylene glycol di(meth)acrylate and dipropylene glycol di(meth)acrylate and various multifunctional (meth)acrylates and multifunctional amine (meth)acrylates. Monofunctional acrylates, i.e., those containing only one acrylate group, may also be advantageously used. Typical monoacrylates include 2-ethylhexyl (meth)acrylate, 2-hydroxyethyl (meth)acrylate, cyanoethyl (meth)acrylate, 2-hydroxypropyl (meth)acrylate, p-dimethyl aminoethyl (meth)acrylate, lauryl (meth)acrylate, cyclohexyl (meth)acrylate, tetrahydrofurfuryl (meth)acrylate, chlorobenzyl (meth)acrylate, amino alkyl(meth)acrylate, various alkyl(meth)acrylates and glycidyl (meth)acrylate. Of course, mixtures of (meth)acrylates or their derivatives as well as combinations of one or more (meth)acrylate monomers, oligomers and/or prepolymers or their derivatives with other copolymerizable monomers, including acrylonitriles and methacrylonitriles may be used as well. Multifunctional (meth)acrylate monomers will typically have at least two, at least three, and preferably at least four, at least five, or even at least six polymerizable functional groups.

For ease of reference in this specification and in the claims, the term “monomer” or “monomers” as used herein with regard to the structural materials that form the wall polymer of the delivery particles is to be understood as monomers, but also is inclusive of oligomers and/or prepolymers formed of the specific monomers.

As used herein the term “water soluble material” means a material that has a solubility of at least 0.5% wt in water at 60° C.

As used herein the term “oil soluble” means a material that has a solubility of at least 0.1% wt in the core of interest at 50° C.

As used herein the term “oil dispersible” means a material that can be dispersed at least 0.1% wt in the core of interest at 50° C. without visible agglomerates.

As used herein, “delivery particles,” “particles,” “encapsulates,” “microcapsules,” and “capsules” are used interchangeably, unless indicated otherwise. As used herein, these terms typically refer to core/shell delivery particles.

### SUMMARY OF THE INVENTION

The invention describes a population of core-shell delivery particles comprising a core material and a shell encapsulating the core material. The core comprises a benefit agent, and the shell comprises a polymeric material. The polymeric material is a reaction product of a cross-linking agent and a modified chitosan. The chitosan is a modified chitosan wherein the chitosan is treated with a redox initiator under acid conditions, leading to unique properties in the polymeric material. The modified chitosan can be further treated with additional acid. “Core-shell encapsulates” and “delivery particles” are used interchangeably when referring to the population of core-shell delivery particles herein.

The compositions of the invention and methods of manufacture, make possible delivery particles which are compatible with finished matrices such as laundry formulations or fabric care formulations. Stability within such matrices is enhanced with the compositions of the invention. Compatibility is ascertained by examination of the extent of agglomeration measured by aggregate particles size increase in representative matrices. The delivery particles of the invention are able to achieve compatibility while also meeting requirements for biodegradability yet having high structural integrity so as to reduce leakage and resist damage to the benefit agent in the core from harsh environments.

The invention teaches improved delivery particles in terms of at least one property category, and preferably more than one property category specifically the categories of leakage, degradability, and compatibility. Compatibility is in terms of computability with a laundry matrix, determined as measured as described herein. In embodiments, delivery particles are described having improved leakage and degradability and compatibility with matrices.

The redox initiator is selected from a persulfate or a peroxide. Preferably, the redox initiator is selected from the group consisting of ammonium persulfate, sodium persulfate, potassium persulfate, cesium persulfate, benzoyl peroxide, hydrogen peroxide, and mixtures thereof. Treatment of chitosan with a redox initiator under acidic conditions modifies chitosan and depolymerizes the chitosan to an average molecular weight of from 1 to 600 kDal., preferably from 5 to 300 kDal., more preferably from 30-100 kDal. Surprisingly, chitosan treated concurrently or sequentially with a redox initiator leads to an unexpected higher performing encapsulate while having enhanced degradability. The acid and redox initiator treatment reduces viscosity making for ease in handling. The combination of treatment with acid and with redox initiator can be accomplished in the water phase or with addition of redox initiator to the emulsion. Chitosan can be modified with redox initiator in the water phase or chitosan can be modified with redox initiator addition to the emulsion, or to both. Chitosan can be acid treated in the water phase followed by modification of the acid treated chitosan in the emulsion. Chitosan can be modified with redox initiator under acidic conditions in the water phase followed by further addition of a redox initiator in the emulsion. Chitosan becomes a modified chitosan when chitosan is treated with a redox initiator.

Surprisingly, the shell of the novel core-shell encapsulate taught herein is degradable at a rate able to meet the requirements of test methods such as OECD 301B. The invention teaches an encapsulate able to degrade at least 40% in 60 days when tested according to test method OECD 301B.

Surprisingly the acid and redox initiator treated delivery particles had better compatibility in matrices such as laundry detergent compared to acid only treated delivery particles.

In some embodiments, the chitosan initially is acid treated, followed by modification with redox initiator to form a modified chitosan. The acid-treated chitosan comprises a hydrolyzate resulting from treatment of chitosan with acid or with a mixture of a first acid and a second acid. The first acid comprises a strong acid, and the second acid comprises a weak acid, wherein the chitosan is treated at a pH of 6.5 or less, or even less than pH 6.5, or even at a pH of from 3 to 6.2, or even at pH of from 5 to 6.2, and a temperature of at least 25° C. for a period of time to obtain a treated chitosan solution with a viscosity of less than 1500 cp, and preferably less than 500 cp viscosity. Such period for treatment typically is for at least one hour. In the process of the invention, the first acid and the second acid are present in a normality ratio from about 20:80 to about 80:20, preferably from about 35:65 to about 65:35. In embodiments, at least 21 wt % of the shell is comprised of moieties derived from acid treated chitosan, further treated with the redox initiator. The first acid is a strong acid and can be selected from the group consisting of hydrochloric, perchloric, nitric, sulfuric and a mixture thereof. Such acid treated chitosan can also be further treated with the redox initiator, forming an acid-treated modified chitosan.

Modified chitosan is formed by treating chitosan with a redox initiator. In various embodiments, this can comprise an acid and redox initiator treated chitosan. The process can comprise forming a hydrolyzate resulting from treatment of chitosan with an acid or a mixture of a first acid and a second acid, and a redox initiator in any order. The redox initiator forms the modified chitosan. The treatment of the chitosan and/or modified chitosan can comprise treating acid, preferably with a first acid comprising a strong acid, and a second acid comprising a weak acid, wherein the chitosan is treated at a pH of 6.5 or less, or even less than pH 6.5, or even at a pH of from 3 to 6.2, or even at pH of from 5 to 6.2, and a temperature of at least 25° C. for a period of time to obtain a treated chitosan solution with a viscosity of less than 1500 cp, and preferably less than 500 cp viscosity. Such period for treatment typically is for at least one hour. In the process of the invention, the first acid and the second acid are present in a normality ratio from about 20:80 to about 80:20, preferably from about 35:65 to about 65:35. In embodiments, at least 21 wt % of the shell is comprised of moieties derived from the chitosan modified with redox initiator, or from the acid-treated and redox initiator modified chitosan, optionally further modified in the emulsion with the same or a different redox initiator. The first acid is a strong acid and can be selected from the group consisting of hydrochloric, perchloric, nitric, sulfuric and a mixture thereof.

The second acid is an organic acid selected from the group consisting of formic acid, acetic acid, ascorbic acid, glutamic acid, lactic acid, maleic acid, malic acid, succinic acid, citric acid, acrylic acid, oxalic acid, tartaric acid, and a mixture thereof.

In embodiments, the first acid has a first pKa of less than 1, and the second acid has a first pKa of 5.5 or less. In that acids can be diprotic or polyprotic, it is to be understood that such acids have a first pKa and additional pKa's based on the additional acid groups. For clarity herein, the first pKa of the respective diprotic or polyprotic acid was used as a selection parameter.

The redox initiator for modifying the chitosan is a persulfate or a peroxide. The redox initiator can be selected from the group consisting of ammonium persulfate, sodium persulfate, potassium persulfate cesium persulfate, benzoyl peroxide, and hydrogen peroxide. The persulfate or peroxide comprises from 0.1 to 99 wt % of the chitosan. The weight ratio of redox initiator to chitosan is from 90/10 to 0.01/99.99, preferably from 50/50 to 1/99, more preferably from 30/70 to 3/97. When persulfate is employed, the sulfate group is believed to ionically bond with the amino group of chitosan. The shells of the delivery particles may comprise sulfur atoms, which can result, for example, from interactions between sulfur-containing redox initiators (e.g., persulfate compounds) and chitosan. The sulfur atoms may be present in the shell at a level of from about 0.1% to about 20%, more preferably from about 0.1% to about 10%, even more preferably from about 0.1% to about 1%, by weight of the shell. The presence and amount of sulfur atoms can be determined by Energy Dispersive X-ray microanalysis according to the EDX Method provided in the Test Method section below.

In the method of making a population of core-shell delivery particles, the core comprises a benefit agent, the shell comprises a polymeric material that is the reaction product of a cross-linking agent, such as polyisocyanate and a modified chitosan or an acid-treated chitosan and a redox initiator. The method comprises providing a water phase by dissolving or dispersing into an aqueous solution, in any order, a chitosan, a redox initiator and a first acid.

The pH of the water phase is adjusted to a pH of 6.5 or less, or even less than pH 6.5, or even at a pH of from 3 to 6.2, or even at pH of from 5 to 6.2, by addition of at least a first acid and a redox initiator, and heating to a temperature of at least 25° C., to form a hydrozylate comprising the chitosan treated with the acid and modified with the redox initiator.

An oil phase is formed comprising the steps of dissolving together at least one benefit agent and at least one polyisocyanate, optionally with an added oil.

An emulsion is formed by mixing under high shear agitation the water phase and the oil phase into an excess of the water phase, thereby forming droplets of the oil phase and benefit agent dispersed in the water phase, and optionally adjusting the pH of the emulsion to be in a range from pH 3 to pH 6. Optionally, a second redox initiator is added to the emulsion either at the milling temperature or at elevated temperature.

The emulsion is cured by heating to at least 40° C., for a time sufficient to form a shell at an interface of the droplets with the water phase, the shell comprising the reaction product of the polyisocyanate, and the acid treated and redox initiator treated chitosan, and the shell surrounding the core comprising the droplets of the oil phase and benefit agent.

Preferably, at least 21 wt % of the shell comprises the acid treated and redox initiator modified chitosan. The redox initiator is selected from a persulfate or a peroxide. A second redox initiator, which is the same or different from first redox initiator, can be added to the emulsion.

Additionally a second acid can be added to the water phase. It can be beneficial to select the first acid as a strong acid and the second acid as a weak acid. Desirably the first acid and the second acid are present in a normality ratio of from about 20:80 to about 80:20, preferably from about 35:65 to about 65:35.

In further constructs, the delivery particles of the invention can be fashioned into new articles by incorporation into various articles of manufacture. Such article can be selected from the group consisting of an agricultural formulation, a slurry encapsulating an agricultural active, a population of dry delivery particles encapsulating an agricultural active, an agricultural formulation encapsulating an insecticide, and an agricultural formulation for delivering a preemergent herbicide. The agricultural active can be selected from the group consisting of an agricultural herbicide, an agricultural pheromone, an agricultural pesticide, an agricultural nutrient, an insect control agent and a plant stimulant.

### BRIEF DESCRIPTION OF THE DRAWINGS

FIG. 1 shows a digital image of delivery particles.

FIG. 2 shows various images associated with the intensity of each peak measured with the EDX method.

FIG. 3 shows a graph of an EDX spectrum for a given sample.

FIG. 4 illustrates delivery particles according to the invention. FIG. 4 depicts the charge difference of encapsulates comparing acid treatment and redox initiator addition to the water phase or to the emulsion as described in the respective examples. As the examples show, the invention enables the zeta potential to be tailored. The invention effects lowering or moderating of the zeta potential at pH conditions of use, yielding a more controllable encapsulate, which usefully is less prone to agglomeration and more compatible with matrices in end use applications.

### DETAILED DESCRIPTION

The invention describes a delivery particle comprising a core material and a shell encapsulating the core material. The core material can comprise a benefit agent. The shell comprises a polymer.

The invention describes compositions that include delivery particles having shells made, at least in part, from chitosan-based materials. More specifically, the shells include chitosan that has been treated with a redox initiator, such as persulfate or peroxide. The chitosan may further be treated with acid. The resulting modified chitosan is then reacted with a cross-linker to form the shells of the delivery particles. “Modified chitosan” is to be understood as chitosan treated with a redox initiator.

The delivery particles have shells made, at least in part, from chitosan-based materials. The shell is a reaction product of a cross-linking agent such as polyisocyanate and an acid-treated chitosan, further treated with a redox initiator such as persulfate or peroxide. The redox initiator forms a modified chitosan. To form the modified chitosan, the redox initiator can be added in the water phase, added to the emulsion or added to both. In particular, the delivery particles include a shell comprising a reaction product of chitosan and a cross-linking agent. In an embodiment, the chitosan is characterized by having been treated with an acid. In an alternate embodiment the acid is a mixture of a first acid and a second acid, the first acid comprising a strong acid, and the second acid comprising a weak acid. The acid treated chitosan is also treated with a redox initiator to form a modified chitosan. The acid treatment is seen to result in an increase within a particular range of the average molecular weight, yet with a surprising reduction in viscosity of the treated chitosan. The redox initiator is seen to depolymerize the chitosan, further reducing the viscosity of the treated chitosan.

In particular the invention comprises a composition comprising a core-shell encapsulate. The core comprises a benefit agent. The shell comprises a polymeric material that is the reaction product of a cross-linking agent, such as polyisocyanate, and a modified chitosan (a chitosan treated with a redox initiator), or an acid treated chitosan along with a redox initiator (a modified chitosan also treated with acid). The redox initiator can be selected from a persulfate or a peroxide. The acid treated chitosan forms a hydrolyzed chitosan.

It is believed that it is also beneficial to treat the chitosan under acidic conditions. The acidic conditions can improve the solubility of the chitosan, thereby making it more available to react with the redox initiator to form the modified chitosan. It is also believed that the acidic conditions can affect the molecular weight and/or structure of the chitosan, leading to improved particles and/or performance.

For example, the modified chitosan may be formed under acidic conditions at a temperature of at least 25° C., preferably at a pH of 6.5 or less, preferably less than 6.5, even more preferably at a pH of from about 3 to about 6.2, more preferably from about 5 to about 6.2.

The chitosan (which, prior to acid treatment and/or modification with redox initiator treatment, may be referred to as raw chitosan or parent chitosan) may preferably be treated at a pH of 6.5 or less with an acid for at least one hour, preferably from about one hour to about three hours, or for a period of time required to obtain a chitosan solution viscosity of not more than about 1500 cps of the acid-treated chitosan, or even not more than 500 cps, at a temperature of from about 25° C. to about 99° C., preferably from about 75° C. to about 95° C.

The modified chitosan (chitosan treated with redox initiator) may be an acid-treated modified chitosan. For example, the chitosan may be treated with an acid. The acid may comprise a weak acid. The acid preferably comprises a mixture of acids, more preferably a mixture of a first acid and a second acid, wherein the first acid is a strong acid, and wherein the second acid is a weak acid. Preferably, the first acid and the second acid are present in a normality ratio of from about 20:80 to about 80:20, preferably from about 35:65 to about 65:35. The first acid may have a first pKa of less than 1, and the second acid may have a first pKa of 5.5 or less. Preferably, the second acid has a first pKa from 1 to 5.5.

The first acid may comprise, consist essentially of, or consist of a strong acid selected from the group consisting of hydrochloric acid, perchloric acid, nitric acid, sulfuric acid, and a mixture thereof, preferably hydrochloric acid. The second acid may comprise, consist essentially of, or consist of a weak acid selected from the group consisting of formic acid, acetic acid, ascorbic acid, glutamic acid, lactic acid, maleic acid, malic acid, succinic acid, citric acid, acrylic acid, oxalic acid, tartaric acid, and a mixture thereof, preferably formic acid, acetic acid, and a mixture thereof.

The chitosan may be treated with an acid prior to modification, i.e., prior to being treated with a redox initiator. However, it may be convenient to treat the chitosan with a redox initiator and an acid simultaneously for at least a portion of the treatment process. For example, the chitosan may be dissolved or dispersed in an acidic water phase, and the redox initiator may be added after dissolution/dispersion. Alternatively, an acid and a redox initiator may be provided to a water phase (in any suitable order), and then chitosan is added and dissolved/dispersed.

It is believed that selecting chitosan and/or modified chitosan with a particular molecular weight can contribute to improved processibility, performance, and/or biodegradability. Chitosan that is relatively too large may result in solutions with high viscosity that are difficult to process. Chitosan that is relatively too small may result in poorer shell formation, likely due to increased solubility of the chitosan, resulting in the chitosan being less likely to migrate to the water/oil interface during shell formation.

The chitosan, prior to treatment with the redox initiator and/or acid, preferably at least prior to treatment with the redox initiator, may be characterized by a weight average molecular weight of from about 100 kDa to about 600 kDa, preferably from about 100 kDa to about 500 kDa, more preferably from about 100 kDa to about 400 kDa, more preferably from about 100 kDa to about 300 kDa, even more preferably from about 100 kDa to about 200 kDa.

The modified chitosan, following treatment with the redox initiator and/or acid, preferably at least following treatment with the redox initiator, may be characterized by a weight average molecular weight of from about 1 kDa to about 600 kDa, preferably from about 5 kDa to about 300 kDa, more preferably from about 10 kDa to about 200 kDa, more preferably from about 15 kDa to about 150 kDa, even more preferably from about 20 kDa to about 100 kDa. The modified chitosan may be characterized by a weight average molecular weight of from about 1 kDa to about 600 kDa, preferably from about 5 kDa to about 300 kDa, more preferably from about 30 kDa to about 100 kDa.

The chitosan may be characterized by a degree of deacetylation of at least 50%, preferably from about 50% to about 99%, more preferably from about 75% to about 90%, even more preferably from about 80% to about 85%. The degree of deacetylation can affect the solubility of the chitosan, which in turn can affect its reactivity or behavior in the process of forming the particle shells. For example, a degree of deacetylation that is too low (e.g., below 50%) results in chitosan that is relatively insoluble and relatively unreactive. A degree of deacetylation that is relatively high can result in chitosan that is very soluble, resulting in relatively little of it traveling to the oil/water interface during shell formation.

The chitosan may further be modified with charged moieties. For example, the chitosan, before or after treatment with the redox initiator, may comprise anionically modified chitosan, cationically modified chitosan, or a combination thereof. Modifying the chitosan in an anionic and/or cationic fashion can change the character of the shell of the delivery particle, for example, by changing the surface charge and/or zeta potential, which can affect the deposition efficiency and/or formulation compatibility of the particles. For example, the modified chitosan may further be modified with a modifying compound, wherein the modifying compound comprises an epoxide, an aldehyde, an α,β-unsaturated compound, or a combination thereof.

The redox initiator modifies the chitosan and depolymerizes chitosan to an average molecular weight of from 1 to 600 kDal. The reduction in molecular weight helps improve the workability of the chitosan by reducing the viscosity. Acid treatment itself increases molecular weight but surprisingly reduces viscosity. The redox initiator reduces viscosity further, beneficially making the material more versatile for use in shell forming encapsulation processes.

The shell of the core-shell encapsulate degrades at least 40% in 60 days when tested according to test method OECD 301B.

The acid-treated modified chitosan results from treatment of chitosan with an acid and modification with a redox initiator, preferably the mixture of a first acid and a second acid. The first acid comprises a strong acid, and the second acid comprises a weak acid. The chitosan is treated at a pH of 6.5 or less, or even less than pH 6.5, or even at a pH of from 3 to 6.2, or even at pH of from 5 to 6.2, and a temperature of at least 25° C. for at least about one hour, more particularly for a period of time to obtain a treated chitosan solution with a viscosity of less than 1500 cp, and preferably less than 500 cp viscosity. Such period for treatment typically is for at least one hour.

The first acid and the second acid are present in a normality ratio from about 20:80 to about 80:20, preferably from about 35:65 to about 65:35. At least 21 wt % of the shell is comprised of moieties derived from the acid treated chitosan.

The first acid is a strong acid selected from the group consisting of hydrochloric, perchloric, nitric, sulfuric and a mixture thereof. The second acid is an organic acid selected from the group consisting of formic acid, acetic acid, ascorbic acid, glutamic acid, lactic acid, maleic acid, malic acid, succinic acid, citric acid, acrylic acid, oxalic acid, tartaric acid, and a mixture thereof.

The first acid has a first pKa of less than 1, and the second acid having a first pKa of 5.5 or less. Preferably the second acid has a first pKa from 1 to 5.5.

The redox initiator is selected from the group consisting of ammonium persulfate, sodium persulfate, and potassium persulfate cesium persulfate, benzoyl peroxide, and hydrogen peroxide. Combinations of initiators can optionally be employed.

The ratio of persulfate or peroxide to raw chitosan is from 0.01/99.99 to 95/5 on the basis of weight.

The cross-linking agent preferably a polyisocyanate is to be understood herein as encompassing monomers, oligomers, and prepolymers selected from any aliphatic or aromatic isocyanates, including by way of illustration and not limitation any of the group consisting of a polyisocyanurate of toluene diisocyanate, a trimethylol propane adduct of toluene diisocyanate, a trimethylol propane adduct of xylylene diisocyanate, methylene diphenyl isocyanate, toluene diisocyanate, [diisocyanato(phenyl)methyl]benzene, tetramethylxylidene diisocyanate, naphthalene-1,5-diisocyanate, phenylene diisocyanate, derivatives thereof, and mixtures thereof.

The core-shell encapsulate has a ratio of core to shell up to 99:1, or even 99.5:0.5, on the basis of weight. The benefit agent is preferably perfume or fragrance, but can be selected from any of the group consisting of perfume, fragrance, agricultural active, phase change material, essential oil, lubricant, colorant, preservative, antimicrobial active, antifungal active, herbicide, antiviral active, antiseptic active, antioxidant, biological active, deodorant, emollient, humectant, exfoliant, ultraviolet absorbing agent, corrosion inhibitor, silicone oil, wax, bleach particle, fabric conditioner, malodor reducing agent, dye, optical brightener, antiperspirant active and mixture thereof.

The core-shell delivery particles can have a median particle size of from 1 to 200 or even to 300 microns. Particle sizes of the encapsules from 1 to 100 microns are preferred.

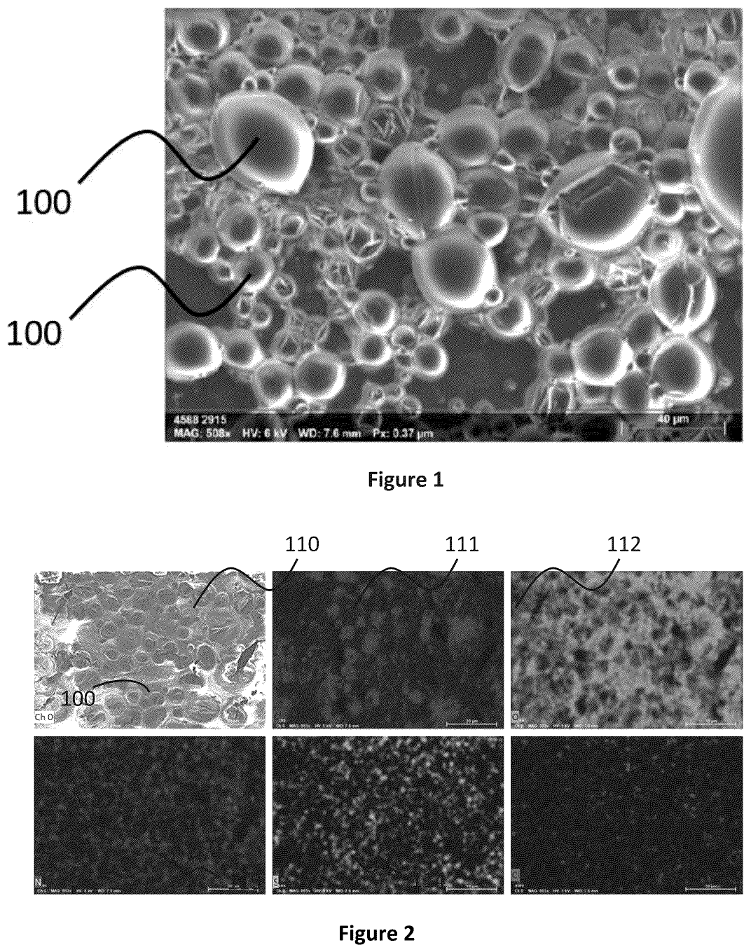

Delivery particles according to the invention, described in the examples, and as shown in FIG. 1, are cationic, with a zeta potential of at least 1 mV or even 15 mV at a pH of 4.5.

For the delivery particles as taught herein, the shell degrades at least 40% of its mass after at least 60 days when tested according to test method OECD 301B.

Without wishing to be bound by theory, it is believed that careful selection of the chitosan's molecular weight can be advantageous. For example, selection of a chitosan having a molecular weight above a certain threshold can result in delivery particles that perform better at certain touchpoints compared to particles made from chitosan of a lower molecular weight. Furthermore, selection of chitosan characterized by a relatively high molecular weight can result in processing challenges, as such chitosan tends to build viscosity, particularly in aqueous environments; the relatively high viscosity can affect the convenient flowability of such solutions and/or inhibit the adequate formation of particle walls. Surprisingly, treatment with acid and redox initiator yields a chitosan wherein the average weight decreases from around 5 kDal to around 300 kDal. Combination with a redox initiator in particular a persulfate was seen to depolymerize the particle wall material further decreasing viscosity and enabling ease of handling.

The chitosan, delivery particles, treatment compositions, and related methods of the present disclosure are discussed in more detail below.

The invention teaches a composition comprising a core-shell encapsulate. The core comprising a benefit agent, preferably a perfume, and the shell comprising a polymeric material that is the reaction product of a cross-linking agent and a modified chitosan. In forming the composition of the invention, chitosan is treated with a mixture of a first acid and a second acid and modified with a redox initiator, the first acid comprising a strong acid, and the second acid comprising a weak acid. The chitosan is treated with the mixture of acids and the redox initiator at a pH of 6.5 or less, or even less than pH 6.5, or even at a pH of from 3 to 6.2, or even at a pH of 5 to 6.2, and a temperature of at least 25° C. for at least one hour, or for a period of time to obtain a treated chitosan solution with a viscosity of less than 1500 cp, and preferably less than 500 cp viscosity. Such period for treatment typically is for at least one hour.

The first acid of the acid mixture and the second acid of the acid mixture are present in a normality ratio from about 20:80 to about 80:20, preferably from about 35:65 to about 65:35. In the composition, desirably at least 21 wt % of the shell is comprised of moieties derived from the acid treated chitosan.

The first acid of the acid mixture is a strong acid selected from the group consisting of hydrochloric, perchloric, nitric, sulfuric and even mixtures thereof. The second acid is an organic acid selected from the group consisting of formic acid, acetic acid, ascorbic acid, glutamic acid, lactic acid, maleic acid, malic acid, succinic acid, citric acid, acrylic acid, oxalic acid, tartaric acid, and also can be mixtures thereof.

The first acid can be selected to have a pKa of less than 1, and the second acid a pKa of 5.5 or less, preferably a pKa from 1 to 5.5. The acids can be monoprotic, diprotic, or polyprotic. It is to be understood that diprotic, triprotic or polyprotic acids will have more than one ionizable hydrogen, and therefore have a first or initial pKa and additional pKa values for the additional ionizable hydrogens, respectively. For purposes hereof, the first pKa refers to the first or initial ionizable hydrogen when the acid is diprotic or polyprotic.

The shell can comprise 1 to 25 percent by weight of the core-shell encapsulate.

The cross-linking agent of the composition can be selected from the group consisting of a polyisocyanurate of toluene diisocyanate, a trimethylol propane adduct of toluene diisocyanate, a trimethylol propane adduct of xylylene diisocyanate, methylene diphenyl diisocyanate, [diisocyanato(phenyl)methyl]benzene, toluene diisocyanate, tetramethylxylidene diisocyanate, naphthalene-1,5-diisocyanate, and phenylene diisocyanate, derivatives thereof and combinations thereof.

When formulated according to the teachings of the invention, the shell degrades at least 40% or even at least 60% of its mass after at least 60 days when tested according to test method OECD 301B.

The core-shell encapsulate has a ratio of core to shell of at least 75:25, or even up to 99:1, or even at least 99.5:0.5, on the basis of weight.

The benefit agent is selected from the group consisting of perfume, fragrance, agricultural active, phase change material, essential oil, lubricant, colorant, preservative, antimicrobial active, antifungal active, herbicide, antiviral active, antiseptic active, antioxidant, biological active, deodorant, emollient, humectant, exfoliant, ultraviolet absorbing agent, corrosion inhibitor, silicone oil, wax, bleach particle, fabric conditioner, malodor reducing agent, dye, optical brightener, antiperspirant active and mixture thereof.

A method of making a population of core-shell delivery particles is also described, the core comprises a benefit agent, the shell comprises a polymeric material that is the reaction product of a cross-linking agent and a modified chitosan, or an acid-treated chitosan and a redox initiator. The method comprises forming a water phase by treating chitosan with an acid, preferably a mixture of a first acid and a second acid and modifying the chitosan with a redox initiator, the first acid comprising a strong acid, and the second acid comprising a weak acid, wherein the chitosan is treated at a pH of 6.5 or less, or even less than pH 6.5, or even at a pH of from 3 to 6.2, or even at pH of from 5 to 6.2, and a temperature of at least 25° C., for at least one hour, or viscosity of 1500 cps or even 500 cps, wherein the first acid and the second acid are present in a normality ratio of from about 20:80 to about 80:20, preferably from about 35:65 to about 65:35, and thereby forming an acid treated chitosan.

The steps further include: forming an oil phase comprising dissolving together at least one benefit agent and at least one cross-linking agent, preferably a polyisocyanate, optionally with an added oil; forming an emulsion by mixing under high shear agitation the water phase and the oil phase into an excess of the water phase, thereby forming droplets of the oil phase and benefit agent dispersed in the water phase, and optionally adjusting the pH of the emulsion to be in a range from pH 3 to pH 6; curing the emulsion by heating to at least 40° C., for a time sufficient to form a shell at an interface of the droplets with the water phase, the shell comprising the reaction product of the cross-linking agent and the acid-treated and redox-initiator-modified chitosan, and the shell surrounding the core comprising the droplets of the oil phase and benefit agent.

A process of making a population of delivery particles is also described wherein the delivery particles comprise a core and a shell surrounding the core. In particular, the process comprises the steps of:
forming a water phase by treating chitosan with a redox initiator in the presence of water at a pH of 6.5 or less and at a temperature of at least 25° C., preferably for at least one hour and/or to a time at which the water phase is characterized by a viscosity of less than 1500 cp, preferably less than 500 cp viscosity, to form a modified chitosan,
forming an oil phase, the forming step comprising dissolving together at least one benefit agent and at least one cross-linking agent, preferably a polyisocyanate, optionally with an added oil, preferably a partitioning modifier;
forming an emulsion by mixing the oil phase into an excess of the water phase, preferably under high shear agitation, thereby forming droplets of the oil phase dispersed in the water phase, and optionally adjusting the pH of the emulsion to be in a range from pH 2 to pH 6;
optionally providing a second redox initiator to the emulsion to further form the modified chitosan.
   The second redox initiator can be the same or different as the redox initiator added to the water phase;
   The emulsion is cured at a temperature of at least 40° C. for a time sufficient to form a shell at an interface of the droplets with the water phase. The shell comprises the reaction product of the cross-linking agent and the modified chitosan. The shell surrounds the core comprising the droplets of the oil phase.

In a variation of the process, a process of making a population of delivery particles is described wherein the delivery particles comprise a core and a shell surrounding the core, the process comprises the steps of:
forming a water phase by treating chitosan in the presence of water at a pH of 6.5 or less and at a temperature of at least 25° C., preferably for at least one hour and/or to a time at which the water phase is characterized by a viscosity of less than 1500 cp, preferably less than 500 cp viscosity, forming an oil phase, the forming step comprising dissolving together at least one benefit agent and at least one cross-linking agent, preferably a polyisocyanate, optionally with an added oil, preferably a partitioning modifier;
forming an emulsion by mixing the oil phase into an excess of the water phase, preferably under high shear agitation, thereby forming droplets of the oil phase dispersed in the water phase, and optionally adjusting the pH of the emulsion to be in a range from pH 2 to pH 6;
adding a redox initiator to the emulsion to form the modified chitosan, and
curing the emulsion at a temperature of at least 40° C. for a time sufficient to form a shell at an interface of the droplets with the water phase.
   The shell comprises the reaction product of the cross-linking agent and the modified chitosan. The shell surrounds the core comprising the droplets of the oil phase.

Unless otherwise noted, all component or composition levels are in reference to the active portion of that component or composition, and are exclusive of impurities, for example, residual solvents or by-products, which may be present in commercially available sources of such components or compositions.

### Shell

To create the delivery particle of the invention a water phase is prepared comprising one or more acids, chitosan and one or more redox initiators. The pH of the water phase is adjusted to a pH of less than 6.5, or even to the range of pH 3 to 6.5. This treatment of chitosan creates a hydrozylate, protonating at least a portion of the amine groups of chitosan and facilitating dissolving in water. Hydrolysis is carried out with heating for a period at an acidic pH such as about 5 or 5.5 or 6. Optionally the redox initiator can be added to the emulsion during formation of the encapsulate, or alternatively, a second redox initiator can be added to the emulsion. Beneficial results were achieved with addition of persulfate to the water phase followed by addition of peroxide to the emulsion. The first and second redox initiators can each be independently selected from persulfate or peroxide.

The modified chitosan comprising redox-initiator-treated chitosan, or the acid treated and redox-initiator-treated chitosan, is used for a reaction with a cross-linking agent, preferably an isocyanate or polyisocyanate. This is accomplished by preparing an oil phase containing the core material comprising a benefit agent and the shell-forming cross-linking agent. An emulsion is formed when the oil phase is combined with the water phase under high shear agitation. The emulsion is heated such as to approximately 60 to 95° C., or even 60 to 80° C., or even to 70 to 80° C. initiating reaction with oil phase cross-linking agent such as isocyanate. As reaction proceeds, a redox initiator can be optionally added to the emulsion. The redox initiator can be the same as the first redox initiator added to the water phase or optionally can be a second redox initiator.

The oil phase is prepared by dissolving an isocyanate such as trimers of xylylene diisocyanate (XDI) or polymers of methylene diphenyl isocyanate (MDI), in oil at 25° C. Diluents, for example isopropyl myristate, may be used to adjust the hydrophilicity of the oil phase. The oil phase is then added into the water phase and milled at high speed to obtain a targeted size. The emulsion is cured in one or more heating steps, such as heating to 40° C. in 30 minutes and holding at 40° C. for 60 minutes. Times and temperatures are approximate. The temperature and time are selected to be sufficient to form and cure a shell at the interface of the droplets of the oil phase with the water continuous phase. For example, the emulsion is heated to 85° C. in 60 minutes and then held at 85° C. for 360 minutes to cure the capsules. The slurry is then cooled to room temperature.

The population of delivery particles according to the present disclosure may be characterized by a volume-weighted median particle size from about 1 to about 100 microns, preferably from about 10 to about 100 microns, preferably from about 15 to about 50 microns, more preferably from about 20 to about 40 microns, even more preferably from about 25 to about 35 microns. For certain compositions, it may be preferred that the population of delivery particles is characterized by a volume-weighted median particle size from about 1 to about 50 microns, preferably from about 5 to about 20 microns, more preferably from about 10 to about 15 microns. Different particle sizes are obtainable by controlling droplet size during emulsification.

The delivery particles may be characterized by a ratio of core to shell up to 99:1, or even 99.5:0.5, on the basis of weight. The shell may be present at a level of from about 1% to about 25%, preferably from about 1% to about 20%, preferably from about 1% to 15%, more preferably from about 5% to about 15%, even more preferably from about 10% to about 15%, even more preferably from about 10% to about 12%, by weight of the delivery particle. The shell may be present at a level of least 1%, preferably at least 3%, more preferably at least 5% by weight of the delivery particle. The shell may be present at a level of up to about 25%, preferably up to about 20%, preferably up to about 15%, more preferably up to about 12%, by weight of the delivery particle.

The delivery particles may be cationic in nature, preferably cationic at a pH of 4.5. The delivery particles may be characterized by a zeta potential of at least 15 millivolts (mV) at a pH of 4.5. The delivery particles can be fashioned to have a zeta potential of at least 15 millivolts (mV) at a pH of 4.5, or even at least 40 mV at a pH of 4.5, or even at least 60 mV at a pH of 4.5. Delivery particles prepared with chitosan typically exhibit positive zeta potentials. Such capsules have improved deposition efficiency on fabrics. At higher pH, the particles may be able to be made nonionic or anionic.

The cross-linking agent, preferable isocyanate or polyisocyanate, useful in the invention is to be understood for purposes hereof as isocyanate monomer, isocyanate oligomer, isocyanate prepolymer, or dimer or trimer of an aliphatic or aromatic isocyanate. All such monomers, prepolymers, oligomers, or dimers or trimers of aliphatic or aromatic isocyanates are intended encompassed by the term “isocyanate” as used herein.

The cross-linking agent can comprise an aliphatic or aromatic monomer, oligomer or prepolymer, usefully of two or more isocyanate functional groups. The isocyanate, for example, can be selected from aromatic toluene diisocyanate and its derivatives used in wall formation for delivery particles, or aliphatic monomer, oligomer or prepolymer, for example, hexamethylene diisocyanate and dimers or trimers thereof, or 3,3,5-trimethyl-5-isocyanatomethyl-1-isocyanato cyclohexane tetramethylene diisocyanate. The polyisocyanate can be selected from 1,3-diisocyanato-2-methylbenzene, hydrogenated MDI, bis(4-isocyanatocyclohexyl) methane, dicyclohexylmethane-4,4′-diisocyanate, and oligomers and prepolymers thereof. This listing is illustrative and not intended to be limiting of the polyisocyanates useful in the invention.

The isocyanates useful in the invention can comprise isocyanate monomers, oligomers or prepolymers, or dimers or trimers thereof, having at least two isocyanate groups. Optimal cross-linking can be achieved with isocyanates having at least three functional groups.

Cross-linking agent for purposes of the invention, is understood as encompassing by way of illustration and not limitation any isocyanate monomer, oligomer, prepolymer or polymer having at least two isocyanate groups and comprising an aliphatic or aromatic moiety in the monomer, oligomer or prepolymer. If aromatic, the aromatic moiety can comprise a phenyl, a toluyl, a xylyl, a naphthyl or a diphenyl moiety, more preferably a toluyl or a xylyl moiety. Aromatic polyisocyanate is understood as a polyisocyanate which comprises at least one aromatic moiety. Aromatic polyisocyanates, for purposes hereof, can include diisocyanate derivatives such as biurets and polyisocyanurates. The polyisocyanate, when aromatic, can be, but is not limited to, methylene diphenyl diisocyanate, [diisocyanato(phenyl)methyl]benzene, toluene diisocyanate, tetramethylxylidene diisocyanate, polyisocyanurate of toluene diisocyanate (commercially available from Bayer under the tradename Desmodur® RC), trimethylol propane-adduct of toluene diisocyanate (commercially available from Bayer under the tradename Desmodur® L75), or naphthalene-1,5-diisocyanate, phenylene diisocyanate, 2,2′-methylenediphenyl diisocyanate, 4,4′-methylenediphenyl diisocyanate, 2,4′-methylenediphenyl diisocyanate, tetramethylxylidene diisocyanate, naphthalene-1,5-diisocyanate, 1,4-phenylene diisocyanate, 1,3-diisocyanatobenzene, or trimethylol propane-adduct of xylylene diisocyanate (commercially available from Mitsui Chemicals under the tradename Takenate® D-110N), and combinations thereof.

Isocyanate, which is aliphatic, is understood as a monomer, oligomer, prepolymer or polymer polyisocyanate which does not comprise any aromatic moiety. Aromatic polyisocyanate is understood as a polyisocyanate which comprises at least one aromatic moiety. There is a preference for aromatic polyisocyanate, however, aliphatic polyisocyanates and blends thereof are useful. Aliphatic polyisocyanates include a trimer of hexamethylene diisocyanate, a trimer of isophorone diisocyanate, a trimethylol propane-adduct of hexamethylene diisocyanate (available from Mitsui Chemicals) or a biuret of hexamethylene diisocyanate (commercially available from Bayer under the tradename Desmodur® N 100).

The capsule shell could also be reinforced using additional co-crosslinkers such as multifunctional amines and/or polyamines such as diethylene triamine (DETA), polyethylene imine, and polyvinyl amine. Acrylates may also be used as additional co-crosslinkers, for example to reinforce the shell.

The polymeric material may be formed in a reaction, where the weight ratio of the chitosan present in the reaction to the cross-linker present in the reaction is from about 1:10 to about 1:0.1. It is believed that selecting desirable ratios of the biopolymer to the cross-linking agent can provide desired ductility benefits, as well as improved biodegradability. It may be preferred that at least 21 wt % of the shell is comprised of moieties derived from chitosan, preferably from acid-treated chitosan. Chitosan as a percentage by weight of the shell may be from about 21% up to about 95% of the shell. The ratio of chitosan in the water phase as compared to the cross-linker, preferably an isocyanate, in the oil phase may be, based on weight, from 21:79 to 90:10, or even from 1:2 to 9:1, or even from 1:1 to 7:1. The polymeric material may be formed in a reaction, where the weight ratio of the chitosan or a derivative thereof (which can include acid-treated chitosan) present in the reaction to the cross-linker present in the reaction is from about 1:10 to about 10:1, preferably from about 1:5 to about 5:1, preferably from about 1:4 to about 5:1, more preferably from about 1:1 to about 5:1, more preferably from about 3:1 to about 5:1. The shell may comprise chitosan at a level of 21 wt % or even greater, preferably from about 21 wt % to about 90 wt %, or even from 21 wt % to 85 wt %, or even 21 wt % to 75 wt %, or 21 wt % to 55 wt % of the total shell being chitosan. The chitosan of this paragraph is preferably modified chitosan as described herein.

### Core

The delivery particles of the present teaching include a benefit agent which comprises one or more ingredients that are intended to be encapsulated. The benefit agent is preferred to be perfume or fragrance but can be selected from a number of different materials such as chromogens and dyes, flavorants, perfumes, sweeteners, fragrances, oils, fats, pigments, cleaning oils, pharmaceuticals, pharmaceutical oils, perfume oils, mold inhibitors, antimicrobial agents, fungicides, bactericides, disinfectants, adhesives, phase change materials, scents, fertilizers, nutrients, and herbicides: by way of illustration and without limitation. The benefit agent and oil comprise the core. The core can be a liquid or a solid. With cores that are solid at ambient temperatures, the wall material can usefully enwrap less than the entire core for certain applications where availability of, for example, an agglomerate core is desired on application. Such uses can include scent release, cleaning compositions, emollients, cosmetic delivery and the like. Where the encapsulate core is phase change material, uses can include such encapsulated materials in mattresses, pillows, bedding, textiles, sporting equipment, medical devices, building products, construction products, HVAC, renewable energy, clothing, athletic surfaces, electronics, automotive, aviation, shoes, beauty care, laundry, and solar energy.

The core constitutes the material encapsulated by the microcapsules. Typically, particularly when the core material is a liquid material, the core material is combined with one or more of the compositions from which the internal wall of the microcapsule is formed or solvent for the benefit agent or partitioning modifier. If the core material can function as the oil solvent in the capsules, e.g., acts as the solvent or carrier for either the wall forming materials or benefit agent, it is possible to make the core material the major material encapsulated, or if the carrier itself is the benefit agent, can be the total material encapsulated. Usually however, the benefit agent is from 0.01 to 99 weight percent of the capsule internal contents, preferably 0.01 to about 65 by weight of the capsule internal contents, and more preferably from 0.1 to about 45% by weight of the capsule internal contents. With certain applications, the core material can be effective even at just trace quantities.

Where the benefit agent is not itself sufficient to serve as the oil phase or solvent, particularly for the wall forming materials, the oil phase can comprise a suitable carrier and/or solvent. In this sense, the oil is optional, as the benefit agent itself can at times be the oil. These carriers or solvents are generally an oil, preferably have a boiling point greater than about 80° C. and low volatility and are non-flammable. Though not limited thereto, they preferably comprise one or more esters, preferably with chain lengths of up to 18 carbon atoms or even up to 42 carbon atoms and/or triglycerides such as the esters of C6 to C12 fatty acids and glycerol. Exemplary carriers and solvents include, but are not limited to: ethyldiphenylmethane; isopropyl diphenylethane; butyl biphenyl ethane; benzylxylene; alkyl biphenyls such as propylbiphenyl and butylbiphenyl; dialkyl phthalates e.g. dibutyl phthalate, dioctylphthalate, dinonyl phthalate and ditridecylphthalate; 2,2,4-trimethyl-1,3-pentanediol diisobutyrate; alkyl benzenes such as dodecyl benzene; alkyl or aralkyl benzoates such as benzyl benzoate; diaryl ethers; di(aralkyl)ethers and aryl aralkyl ethers; ethers such as diphenyl ether, dibenzyl ether and phenyl benzyl ether; liquid higher alkyl ketones (having at least 9 carbon atoms); alkyl or aralkyl benzoates, e.g., benzyl benzoate; alkylated naphthalenes such as dipropylnaphthalene; partially hydrogenated terphenyls; high-boiling straight or branched chain hydrocarbons; alkaryl hydrocarbons such as toluene; vegetable and other crop oils such as canola oil, soybean oil, corn oil, sunflower oil, cottonseed oil, lemon oil, olive oil and pine oil; methyl esters of fatty acids derived from transesterification of vegetable and other crop oils, methyl ester of oleic acid, esters of vegetable oil, e.g. soybean methyl ester, straight chain paraffinic aliphatic hydrocarbons, and mixtures of the foregoing.

Useful benefit agents include perfume raw materials, such as alcohols, ketones, aldehydes, esters, ethers, nitriles, alkenes, fragrances, fragrance solubilizers, essential oils, phase change materials, lubricants, colorants, cooling agents, preservatives, antimicrobial or antifungal actives, herbicides, antiviral actives, antiseptic actives, antioxidants, biological actives, deodorants, emollients, humectants, exfoliants, ultraviolet absorbing agents, self-healing compositions, corrosion inhibitors, sunscreens, silicone oils, waxes, hydrocarbons, higher fatty acids, essential oils, lipids, skin coolants, vitamins, sunscreens, antioxidants, glycerine, catalysts, bleach particles, silicon dioxide particles, malodor reducing agents, dyes, brighteners, antibacterial actives, antiperspirant actives, cationic polymers and mixtures thereof. Phase change materials useful as benefit agents can include, by way of illustration and not limitation, paraffinic hydrocarbons having 13 to 28 carbon atoms, various hydrocarbons such n-octacosane, n-heptacosane, n-hexacosane, n-pentacosane, n-tetracosane, n-tricosane, n-docosane, n-heneicosane, n-eicosane, n-nonadecane, octadecane, n-heptadecane, n-hexadecane, n-pentadecane, n-tetradecane, n-tridecane. Phase change materials can alternatively, optionally in addition include crystalline materials such as 2,2-dimethyl-1,3-propanediol, 2-hydroxymethyl-2-methyl-1, 3-propanediol, acids of straight or branched chain hydrocarbons such as eicosanoic acid and esters such as methyl palmitate, fatty alcohols and mixtures thereof.

Preferably, in the case of fragrances, a perfume oil acts as benefit agent and solvent for the wall forming material, as illustrated in the examples herein.

Optionally the water phase may include an emulsifier. Non-limiting examples of emulsifiers include water-soluble salts of alkyl sulfates, alkyl ether sulfates, alkyl isothionates, alkyl carboxylates, alkyl sulfosuccinates, alkyl succinamates, alkyl sulfate salts such as sodium dodecyl sulfate, alkyl sarcosinates, alkyl derivatives of protein hydrolyzates, acyl aspartates, alkyl or alkyl ether or alkylaryl ether phosphate esters, sodium dodecyl sulphate, phospholipids or lecithin, or soaps, sodium, potassium or ammonium stearate, oleate or palmitate, alkylarylsulfonic acid salts such as sodium dodecylbenzenesulfonate, sodium dialkylsulfosuccinates, dioctyl sulfosuccinate, sodium dilaurylsulfosuccinate, poly(styrene sulfonate) sodium salt, isobutylene-maleic anhydride copolymer, gum arabic, sodium alginate, carboxymethylcellulose, cellulose sulfate and pectin, poly(styrene sulfonate), isobutylene-maleic anhydride copolymer, carrageenan, sodium alginate, pectic acid, tragacanth gum, almond gum and agar; semi-synthetic polymers such as carboxymethyl cellulose, sulfated cellulose, sulfated methylcellulose, carboxymethyl starch, phosphated starch, lignin sulfonic acid; and synthetic polymers such as maleic anhydride copolymers (including hydrolyzates thereof), polyacrylic acid, polymethacrylic acid, acrylic acid butyl acrylate copolymer or crotonic acid homopolymers and copolymers, vinyl benzenesulfonic acid or 2-acrylamido-2-methylpropanesulfonic acid homopolymers and copolymers, and partial amide or partial ester of such polymers and copolymers, carboxy modified polyvinyl alcohol, sulfonic acid-modified polyvinyl alcohol and phosphoric acid-modified polyvinyl alcohol, phosphated or sulfated tristyrylphenol ethoxylates, palmitamidopropyltrimonium chloride (Varisoft PATC™, available from Degussa Evonik, Essen, Germany), distearyl diammonium chloride, cetyltrimethylammonium chloride, quaternary ammonium compounds, fatty amines, aliphatic ammonium halides, alkyldimethylbenzylammonium halides, alkyldimethylethylammonium halides, polyethyleneimine, poly(2-dimethylamino)ethyl methacrylate) methyl chloride quaternary salt, poly(1-vinylpyrrolidone-co-2-dimethylaminoethyl methacrylate), poly(acrylamide-co-diallyldimethylammonium chloride), poly(allylamine), poly[bis(2-chloroethyl) ether-alt-1,3-bis[3-(dimethylamino)propyl]urea] quaternized, and poly(dimethylamine-co-epichlorohydrin-co-ethylenediamine), condensation products of aliphatic amines with alkylene oxide, quaternary ammonium compounds with a long-chain aliphatic radical, e.g. distearyldiammonium chloride, and fatty amines, alkyldimethylbenzylammonium halides, alkyldimethylethylammonium halides, polyalkylene glycol ether, condensation products of alkyl phenols, aliphatic alcohols, or fatty acids with alkylene oxide, ethoxylated alkyl phenols, ethoxylated aryl phenols, ethoxylated polyaryl phenols, carboxylic esters solubilized with a polyol, polyvinyl alcohol, polyvinyl acetate, or copolymers of polyvinyl alcohol polyvinyl acetate, polyacrylamide, poly(N-isopropylacrylamide), poly(2-hydroxypropyl methacrylate), poly(-ethyl-2-oxazoline), poly(2-isopropenyl-2-oxazoline-co-methyl methacrylate), poly(methyl vinyl ether), and polyvinyl alcohol-co-ethylene), and cocoamidopropyl betaine. Emulsifier, if employed, is typically from about 0.1 to 40% by weight, preferably 0.2 to about 15% by weight, more typically 0.5 to 10% be weight, based on total weight of the formulation

The delivery particles may encapsulate a partitioning modifier in addition to the benefit agent. Non-limiting examples of partitioning modifiers include isopropyl myristate, mono-, di-, and tri-esters of C₄-C₂₄ fatty acids, castor oil, mineral oil, soybean oil, hexadecanoic acid, methyl ester isododecane, isoparaffin oil, polydimethylsiloxane, brominated vegetable oil, and combinations thereof. Delivery particles may also have varying ratios of the partitioning modifier to the benefit agent so as to make different populations of delivery particles that may have different bloom patterns. Such populations may also incorporate different perfume oils so as to make populations of delivery particles that display different bloom patterns and different scent experiences. Patent publication US 2011-0268802 discloses other non-limiting examples of delivery particles and partitioning modifiers and is hereby incorporated by reference.

Optionally, if desired, the delivery particles can be dewatered such as through decanting, filtration, centrifuging or other separation technique. Alternatively, the aqueous slurry delivery particles can be spray dried.

In some examples of the process and compositions, the delivery particles may consist of one or more distinct populations. The composition may have at least two different populations of delivery particles that vary in the exact make-up of the perfume oil and in the median particle size and/or partitioning modifier to perfume oil (PM:PO) weight ratio. In some examples, the composition includes more than two distinct populations that vary in the exact make up the perfume oil and in their fracture strengths. In some further examples, the populations of delivery particles can vary with respect to the weight ratio of the partitioning modifier to the perfume oil(s). In some examples, the composition can include a first population of delivery particles having a first ratio that is a weight ratio of from 2:3 to 3:2 of the partitioning modifier to a first perfume oil and a second population of delivery particles having a second ratio that is a weight ratio of less than 2:3 but greater than 0 of the partitioning modifier to a second perfume oil.

In some embodiments, each distinct population of delivery particles is preparable in a distinct slurry. For example, the first population of delivery particles can be contained in a first slurry and the second population of delivery particles contained in a second slurry. It is to be appreciated that the number of distinct slurries for combination is without limit and a choice of the formulator such that 3, 10, or 15 distinct slurries may be combined. The first and second populations of delivery particles may vary in the exact makeup of the benefit agent, such as the perfume oil, and in the median particle size and/or PM:PO weight ratio.

In some embodiments, the composition, can be prepared by combining the first and second slurries with at least one adjunct ingredient and optionally packaged in a container. In some examples, the first and second populations of delivery particles can be prepared in distinct slurries and then spray dried to form a particulate. The distinct slurries may be combined before spray drying, or spray dried individually and then combined together when in particulate powder form. Once in powder form, the first and second populations of delivery particles may be combined with an adjunct ingredient to form the composition useful as a feedstock for manufacture of consumer, industrial, medical or other goods. In some examples, at least one population of delivery particles is spray dried and combined with a slurry of a second population of delivery particles. In some examples, at least one population of delivery particles is dried, prepared by spray drying, fluid bed drying, tray drying, or other such drying processes that are available.

In some examples, the slurry or dry particulates can include one or more adjunct materials such as processing aids selected from the group consisting of a carrier, an aggregate inhibiting material, a deposition aid, a particle suspending polymer, and mixtures thereof. Non-limiting examples of aggregate inhibiting materials include salts that can have a charge-shielding effect around the particle, such as magnesium chloride, calcium chloride, magnesium bromide, magnesium sulfate, and mixtures thereof. Non-limiting examples of particle suspending polymers include polymers such as xanthan gum, carrageenan gum, guar gum, shellac, alginates, chitosan; cellulosic materials such as carboxymethyl cellulose, hydroxypropyl methyl cellulose, cationically charged cellulosic materials; polyacrylic acid; polyvinyl alcohol; hydrogenated castor oil; ethylene glycol distearate; and mixtures thereof.

In some embodiments, the slurry can include one or more processing aids, selected from the group consisting of water, aggregate inhibiting materials such as divalent salts; particle suspending polymers such as xanthan gum, guar gum, carboxy methyl cellulose.

In other examples of the invention, the slurry can include one or more carriers selected from the group consisting of polar solvents, including but not limited to, water, ethylene glycol, propylene glycol, polyethylene glycol, glycerol; nonpolar solvents, including but not limited to, mineral oil, perfume raw materials, silicone oils, hydrocarbon paraffin oils, and mixtures thereof.

In some examples, said slurry may include a deposition aid that may comprise a polymer selected from the group comprising: polysaccharides, in one aspect, cationically modified starch and/or cationically modified guar; polysiloxanes; poly diallyl dimethyl ammonium halides; copolymers of poly diallyl dimethyl ammonium chloride and polyvinyl pyrrolidone; a composition comprising polyethylene glycol and polyvinyl pyrrolidone; acrylamides; imidazoles; imidazolinium halides; polyvinyl amine; copolymers of poly vinyl amine and N-vinyl formamide; polyvinyl formamide, polyvinyl alcohol; polyvinyl alcohol crosslinked with boric acid; polyacrylic acid; polyglycerol ether silicone cross-polymers; polyacrylic acids, polyacrylates, copolymers of polyvinylamine and polvyinylalcohol oligomers of amines, in one aspect a diethylenetriamine, ethylene diamine, bis(3-aminopropyl)piperazine, N,N-Bis-(3-aminopropyl)methylamine, tris(2-aminoethyl)amine and mixtures thereof; polyethyleneimine, a derivatized polyethyleneimine, in one aspect an ethoxylated polyethyleneimine; a polymeric compound comprising, at least two moieties selected from the moieties consisting of a carboxylic acid moiety, an amine moiety, a hydroxyl moiety, and a nitrile moiety on a backbone of polybutadiene, polyisoprene, polybutadiene/styrene, polybutadiene/acrylonitrile, carboxyl-terminated polybutadiene/acrylonitrile or combinations thereof; pre-formed coacervates of anionic surfactants combined with cationic polymers; polyamines and mixtures thereof.

In some additional examples to illustrate the invention, at least one population of delivery particles can be contained in an agglomerate and then combined with a distinct population of delivery particles and at least one adjunct material. Said agglomerate may comprise materials selected from the group consisting of silicas, citric acid, sodium carbonate, sodium sulfate, sodium chloride, and binders such as sodium silicates, modified celluloses, polyethylene glycols, polyacrylates, polyacrylic acids, zeolites and mixtures thereof.

Suitable equipment for use in the processes disclosed herein may include continuous stirred tank reactors, homogenizers, turbine agitators, recirculating pumps, paddle mixers, plough shear mixers, ribbon blenders, vertical axis granulators and drum mixers, both in batch and, where available, in continuous process configurations, spray dryers, and extruders. Such equipment can be obtained from Lodige GmbH (Paderborn, Germany), Littleford Day, Inc. (Florence, Ky., U.S.A.), Forberg AS (Larvik, Norway), Glatt Ingenieurtechnik GmbH (Weimar, Germany), Niro (Soeborg, Denmark), Hosokawa Bepex Corp. (Minneapolis, Minn., U.S.A.), Arde Barinco (New Jersey, U.S.A.).

### Test Methods

It is understood that the test methods disclosed in the Test Methods section of the present application should be used to determine the respective values of the parameters of Applicant's claimed subject matter as claimed and described herein.

### Determination of a Polymer's Molecular Weight and Related Parameters

The following method describing gel permeation chromatograph with multi-angle light scatter and refractive index detection (GPC-MALS/RI) is used to find molecular weight distribution measurements and related values of the polymers described herein.

Gel Permeation Chromatography (GPC) with Multi-Angle Light Scattering (MALS) and Refractive Index (RI) Detection (GPC-MALS/RI) permits the measurement of absolute molecular weight of a polymer without the need for column calibration methods or standards. The GPC system allows molecules to be separated as a function of their molecular size. MALS and RI allow information to be obtained on the number average (Mn) and weight average (Mw) molecular weight.

The Mw distribution of water-soluble polymers like chitosan is typically measured by using a Liquid Chromatography system (e.g., Agilent 1260 Infinity pump system with OpenLab Chemstation software, Agilent Technology, Santa Clara, CA, USA) and a column set (e.g., 2 Tosoh TSKgel G6000WP 7.8×300 mm 13 um pore size, guard column A0022 6 mm×40 mm PW xl-cp, King of Prussia, PA) which is operated at 40° C. The mobile phase is 0.1M sodium nitrate in water containing 0.02% sodium azide and 0.2% acetic acid. The mobile phase solvent is pumped at a flow rate of 1 mL/min, isocratically. A multiangle light scattering (18-Angle MALS) detector DAWN® and a differential refractive index (RI) detector (Wyatt Technology of Santa Barbara, Calif., USA) controlled by Wyatt Astra® software v8.0 are used.

A sample is typically prepared by dissolving chitosan materials in the mobile phase at ˜1 mg per ml and by mixing the solution for overnight hydration at room temperature. The sample is filtered through a 0.8 μm Versapor membrane filter (PALL, Life Sciences, NY, USA) into the LC autosampler vial using a 3-ml syringe before the GPC analysis.

A dn/dc value (differential change of refractive index with concentration, 0.15) is used for the number average molecular weight (Mn), weight average molecular weight (Mw), Z-average molecular weight (Mz), molecular weight of the peak maxima (Mp), and polydispersity (Mw/Mn) determination by the Astra detector software.

An illustrative example of these points on a hypothetical graph of a polymer's molecular weight distribution is shown in FIG. 1, where: Mn is indicated with structure number 1; Mp is indicated with structure number 2; Mw is indicated with structure number 3; and Mz is indicated with structure number 4.

### Viscosity

Viscosity of liquid finished product is measured using an AR 550 rheometer/viscometer from TA instruments (New Castle, DE, USA), using parallel steel plates of 40 mm diameter and a gap size of 500 μm. The high shear viscosity at 20 s⁻¹ and low shear viscosity at 0.05 s⁻¹ is obtained from a logarithmic shear rate sweep from 0.01 s⁻¹ to 25 s⁻¹ in 3 minutes time at 21° C.

### Test Method for Determining log P

The value of the log of the Octanol/Water Partition Coefficient (log P) is computed for each material (e.g., each PRM in the perfume mixture) being tested. The log P of an individual material (e.g., a PRM) is calculated using the Consensus log P Computational Model, version 14.02 (Linux) available from Advanced Chemistry Development Inc. (ACD/Labs) (Toronto, Canada) to provide the unitless log P value. The ACD/Labs' Consensus log P Computational Model is part of the ACD/Labs model suite.

### Volume-Weighted Particle Size and Size Distribution

The volume-weighted particle size distribution is determined via single-particle optical sensing (SPOS), also called optical particle counting (OPC), using the AccuSizer 780 AD instrument and the accompanying software CW788 version 1.82 (Particle Sizing Systems, Santa Barbara, California, U.S.A.), or equivalent. The instrument is configured with the following conditions and selections: Flow Rate=1 ml/sec; Lower Size Threshold=0.50 μm; Sensor Model Number=Sensor Model Number=LE400-05 or equivalent; Autodilution=On; Collection time=60 sec; Number channels=512; Vessel fluid volume=50 ml; Max coincidence=9200. The measurement is initiated by putting the sensor into a cold state by flushing with water until background counts are less than 100. A sample of delivery capsules in suspension is introduced, and its density of capsules adjusted with DI water as necessary via autodilution to result in capsule counts of at least 9200 per ml. During a time period of 60 seconds the suspension is analyzed. The resulting volume-weighted PSD data are plotted and recorded, and the values of the desired volume-weighted particle size (e.g., the median/50^{th} percentile, 5^{th} percentile, and/or 90^{th} percentile) are determined.

### Procedure for Determination of % Degradation

% degradation is determined by the “OECD Guideline for Testing of Chemicals” 301B CO₂ Evolution (Modified Sturm Test), adopted 17 Jul. 1992. For ease of reference, this test method is referred to herein as test method OECD 301B

### Procedure for Determination of Free Oil

This method measures the amount of oil in the water phase and uses as an internal standard solution 1 mg/ml dibutyl phthalate (DBP)/hexane.

Weigh a little more than 250 mgs of DBP into a small beaker and transfer to a 250 ml volumetric rinsing the beaker thoroughly. Fill with hexane to 250 ml.

Sample Prep: Weigh approximately 1.5-2 grams (40 drops) of the capsule slurry into a 20 ml scintillation vial and add 10 ml's of the ISTD solution, cap tightly. Shaking vigorously several times over 30 minutes, pipette solution into an autosampler vial and analyze by GC.

Additional details. Instrumentation: HP5890 GC connected to HP Chem Station Software; Column: 5 m×0.32 mm id with 1 μm DB-1 liquid phase; Temperature 50° C.; for 1 minute then heat to 320° C.; @15 deg/min; Injector: 275° C.; Detector: 325° C.; 2 ul injection.

Calculation: Add total peak area minus the area for the DBP for both the sample and calibration.
i) Calculate mg of free core oil:

$$\frac{Total area from sample}{Total are from calibration} \times mg of oil in calibration solution = mg of free oil$$

ii) Calculate % free core oil

$$\frac{mg of free core oil}{Sample wt . ( mg )} \times 100 = \% free core oil in wet slurry$$

### Procedure for Determination of Benefit Agent Leakage

Obtain 2, one-gram samples of benefit agent particle composition. Add 1 gram (Sample 1) of particle composition to 99 grams of product matrix in which the particle will be employed. Age the particle containing product matrix (Sample 1) for 2 weeks at 35° C. in a sealed glass jar. The other one-gram sample (Sample 2) is similarly aged.

After 2 weeks, use filtration to recover the particle composition's particles from the product matrix (Sample 1) and from the particle composition (Sample 2). Treat each particle sample with a solvent that will extract all the benefit agent from each samples' particles. Inject the benefit agent containing solvent from each sample into a Gas Chromatograph and integrate the peak areas to determine the total quantity of benefit agent extracted from each sample.

Determine the percentage of benefit agent leakage by calculating the difference in the values obtained for the total quantity of benefit agent extracted from Sample 2 minus Sample 1, expressed as a percentage of the total quantity of benefit agent extracted from Sample 2, as represented in the equation below:

$$\frac{Sample 2 - Sample 1}{Sample 2} \times 100 = Percentage of Benefit Agent Leakage$$

Delivery particles can be prepared that exhibit positive zeta potentials. Such capsules have improved deposition efficiency, such as on fabrics.

### Sample Preparation for Biodegradability Measurements

The water soluble or water dispersible material is purified via crystallization till a purity of above 95% is achieved and dried before biodegradability measurement.

The oily medium comprising the benefit agent needs to be extracted from the delivery particle slurry in order to only analyze the polymer wall. Therefore, the delivery particle slurry is freeze dried to obtain a powder. Then, it is further washed with organic solvents via Soxhlet extraction method to extract the oily medium comprising the benefit agent till weight percentage of oily medium is below 5% based on total delivery particle polymer wall. Finally, the polymer wall is dried and analyzed.

Weight ratio of delivery particle to solvent is 1:3. Residual oily medium is determined by thermogravimetric analysis (60 minutes isotherm at 100° C. and another 60 minutes isotherm at 250° C.). The weight loss determined needs to be below 5%.

### OECD 301 B—Biodegradability Method

Accumulative CO₂ release is measured over 60 days following the guidelines of the Organisation for Economic Cooperation and Development (OECD)—OECD (1992), Test No. 301: Ready Biodegradability, OECD Guidelines for the Testing of Chemicals, Section 3, OECD Publishing, Paris, https://doi.org/10.1787/9789264070349-en.

### Leakage

The amount of benefit agent leakage from the benefit agent containing delivery particles is determined according to the following method:
i) Obtain two 1 g samples of the raw material slurry of benefit agent containing delivery particles.
ii) Add 1 g of the raw material slurry of benefit agent containing delivery particles to 99 g of the consumer product matrix in which the particles will be employed and label the mixture as Sample 1. Immediately use the second 1 g sample of raw material particle slurry in Step d below, in its neat form without contacting consumer product matrix, and label it as Sample 2.
iii) Age the delivery particle-containing product matrix (Sample 1) for 1 week at 35° C. in a sealed glass jar.
iv) Using filtration, recover the particles from both samples. The particles in Sample 1 (in consumer product matrix) are recovered after the aging step. The particles in Sample 2 (neat raw material slurry) are recovered at the same time that the aging step began for sample 1.
v) Treat the recovered particles with a solvent to extract the benefit agent materials from the particles.
vi) Analyze the solvent containing the extracted benefit agent from each sample, via chromatography.
vii) Integrate the resultant benefit agent peak areas under the curve and sum these areas to determine the total quantity of benefit agent extracted from each sample.
viii) Determine the percentage of benefit agent leakage by calculating the difference in the values obtained for the total quantity of benefit agent extracted from Sample 2 (S2) minus Sample 1 (S1), expressed as a percentage of the total quantity of benefit agent extracted from Sample 2 (s2), as represented in the equation below:

$$\% Leakage = ( \frac{S 2 - S 1}{S 2} ) \times 10$$

### Volume Weighted Median Particle Size

Particle size is measured using static light scattering devices, such as an Accusizer 780A, made by Particle Sizing Systems, Santa Barbara Calif The instrument is calibrated from 0 to 300μ using Duke particle size standards. Samples for particle size evaluation are prepared by diluting about 1 g emulsion, if the volume weighted median particle size of the emulsion is to be determined, or 1 g of benefit agent containing delivery particles slurry, if the finished particles volume weighted median particle size is to be determined, in about 5 g of de-ionized water and further diluting about 1 g of this solution in about 25 g of water.

About 1 g of the most dilute sample is added to the Accusizer and the testing initiated, using the autodilution feature. The Accusizer should be reading in excess of 9200 counts/second. If the counts are less than 9200 additional sample should be added. The Accusizer will dilute the test sample until 9200 counts/second and initiate the evaluation. After 2 minutes of testing the Accusizer will display the results, including volume-weighted median size.

The broadness index can be calculated by determining the particle size at which 95% of the cumulative particle volume is exceeded (95% size), the particle size at which 5% of the cumulative particle volume is exceeded (5% size), and the median particle size (50% size—50% of the particle volume both above and below this size). Broadness Index=((95% size)−(5% size)/50% size).

All temperatures herein are in degrees Celsius (° C.) unless otherwise indicated. Unless otherwise specified, all measurements herein are conducted at 20° C. and under the atmospheric pressure.

All percentages and ratios are calculated by weight unless otherwise indicated. All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Procedure for Measuring Compatibility of Delivery Particles in Laundry Matrix

The compatibility of the delivery particles in laundry matrix is measured by the percentage of the aggregates formed in the laundry detergent matrix. The slurry containing the delivery particles were homogenized by agitation for at least one minutes with an overhead mixer. The homogenized slurry was then added in laundry matrix, such as single unit does (SUD) matrix at a ratio of 1:40, such as 1 g slurry in 40 g matrix, under mixing. Mixing the above mixture for at least 15 minutes at 350 rpm using overhead mixer. The mixture of the delivery particle and laundry matrix was then poured through the 425 μm sieve after mixing. Wash the particle aggregates on the sieve with plenty of deionized (DI) water until no visible laundry matrix is observed. Collect the original and water wash filtrate and then pass through the 212 um sieve to collect any particle aggregates on the 212 um sieve. The particle aggregates were then washed with plenty of DI water until no visible matrix is observed. Combine the particle aggregates from 212 um and 425 um sieve and wash with DI water again to rinse off any remaining matrix. The particle aggregates were then collected and dried in CEM oven to a constant weight to determine the weight of the particle aggregates in laundry matrix. The percent aggregation is calculated by following:

$$\frac{Dry weight of particle aggregates}{( W eight of slurry ) \times ( \% Solid of the slurry )} \times 100 = Percentage aggregate$$

### EDX Method

Energy Dispersive X-ray (EDX) microanalysis is an x-ray technique used to identify the elemental composition of materials. The technique can be qualitative or quantitative and can even provide spatial distribution of elements through mapping because elemental concentrations can be collected from points, along lines, or as maps.

The instrument used in the method as described herein is a Scanning Electron Microscope (SEM) ZEISS 300 equipped with a Bruker Quantax 400 EDX detector.

To analyse delivery particles in a premix or slurry, a 2 μl solution of slurry is deposited on a SEM stub (sample holder) which has previously been cleaned well using acetone and alcohol in sequence.

To analyse delivery particles in a product composition, particles may be extracted according to the “Extraction of delivery particles from finished products” method provided below.

The EDX detector is used according to the manufacturer's instructions to collect the desired data, using the guidance for qualitative and quantitative analysis given below.

The data generated by EDX analysis includes spectra reported in a graph where the x-axis relates to reported X-ray Energy (keV) and the y-axis relates to the intensity of the signal. The graph is characterized by different peaks, each corresponding to the characteristic energy of the detected elements, which subsequently enables definition of the chemical composition of the sample being analysed.

### A. Qualitative Analysis

For a given sample, an elemental mapping is realized to identify the surface arrangement of the detected elements acquiring an area of 140×95 um (corresponding to a magnification of 800×), using a resolution of 600×400 pixel for 3 minutes.

The chemical information produced by the EDX technique can be visualized in several ways including elemental mapping. For a specific region of interest (ROT), a digital image can be acquired where the intensity of each position (pixel) is proportional to the intensity of each peak. FIG. 1 shows a digital image of a specific ROI, using a delivery particle slurry sample; numerous delivery particles 100 are shown. FIG. 2 shows various images (originally in color) associated with the intensity of each peak. Typically, the images are in color, and brighter colors are associated with the greater peak intensity. In FIG. 2, the first image 110 shows a representative sample of delivery particles 100. The second image 111 shows an image representing the carbon that is present. The third image 112 shows an image representing the oxygen that is present. The fourth image 113 shows an image representing the nitrogen that is present. The fifth image 114 shows an image representing the sulfur that is present. The sixth image 115 shows an image representing the chlorine that is present.

### B. Quantitative Analysis

The EDX technique can be used to detect the presence of elements, as well as their concentration. The MDL (Minimum Detection Limit) of this analytical technique is about 0.1 wt % for quantification element; if the mass concentration is lower than the MDL, the element is not quantified.

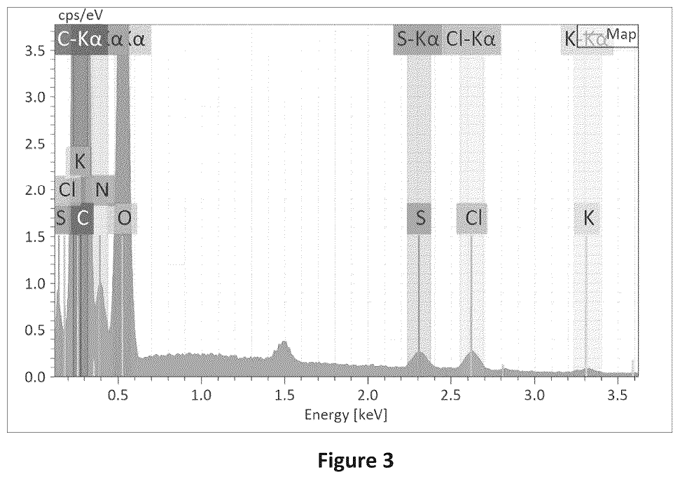

For the quantitative analysis, an EDX spectrum is acquired in an area of 50 um×40 um for 3 minutes. The output is a spectrum, where the peaks are identified as corresponding to the detected elements; a table is also generated that shows mass percentages and atomic distribution percentages (stoichiometric ratio). FIG. 3 shows a graph of a spectrum for a given sample.

Extraction of Delivery Particles from Finished Products

Except where otherwise specified herein, the preferred method to isolate delivery particles from finished products is based on the fact that the density of most such delivery particles is different from that of water. The finished product is mixed with water in order to dilute and/or release the delivery particles. The diluted product suspension is centrifuged to speed up the separation of the delivery particles. Such delivery particles tend to float or sink in the diluted solution/dispersion of the finished product. Using a pipette or spatula, the top and bottom layers of this suspension are removed and undergo further rounds of dilution and centrifugation to separate and enrich the delivery particles. The delivery particles are observed using an optical microscope equipped with crossed-polarized filters or differential interference contrast (DIC), at total magnifications of 100× and 400×. The microscopic observations provide an initial indication of the presence, size, quality and aggregation of the delivery particles.

For extraction of delivery particles from a liquid fabric enhancer finished product conduct the following procedure:
1. Place three aliquots of approximately 20 ml of liquid fabric enhancer into three separate 50 ml centrifuge tubes and dilute each aliquot 1:1 with DI water (e.g. 20 ml fabric enhancer+20 ml DI water), mix each aliquot well and centrifuge each aliquot for 30 minutes at approximately 10000×g.
2. After centrifuging per Step 1, discard the bottom water layer (around 10 ml) in each 50 ml centrifuge tube then add 10 ml of DI water to each 50 ml centrifuge tube.
3. For each aliquot, repeat the process of centrifuging, removing the bottom water layer and then adding 10 ml of DI water to each 50 ml centrifuge tube two additional times.
4. Remove the top layer with a spatula or a pipette, and
5. Transfer this top layer into a 1.8 ml centrifuge tube and centrifuge for 5 minutes at approximately 20000×g.
6. Remove the top layer with a spatula and transfer into a new 1.8 ml centrifuge tube and add DI water until the tube is completely filled, then centrifuge for 5 minutes at approximately 20000×g.
7. Remove the bottom layer with a fine pipette and add DI water until tube is completely filled and centrifuge for 5 minutes at approximately 20000×g.
8. Repeat step 7 for an additional 5 times (6 times in total).

If both a top layer and a bottom layer of enriched delivery particles appear in the above described step 1, then, immediately move to step 3 (i.e., omit step 2) and proceed steps with steps 4 through 8. Once those steps have been completed, also remove the bottom layer from the 50 ml centrifuge tube from step 1, using a spatula or/and a pipette. Transfer the bottom layer into a 1.8 ml centrifuge tube and centrifuge 5 min at approximately 20000×g. Remove the bottom layer in a new tube and add DI water until the tube is completely filled then centrifuge for 5 minutes approximately 20000×g. Remove the top layer (water) and add DI water again until the tube is full. Repeat this another 5 times (6 times in total). Recombine the delivery particle enriched and isolated top and bottom layers back together.

If the fabric enhancer has a white color or is difficult to distinguish the delivery particle enriched layers add 4 drops of dye (such as Liquitint Blue JH 5% premix from Milliken & Company, Spartanburg, South Carolina, USA) into the centrifuge tube of step 1 and proceed with the isolation as described.

For extraction of delivery particles from solid finished products that disperse readily in water, mix 1 L of DI water with 20 g of the finished product (e.g. detergent foams, films, gels and granules; or water-soluble polymers; soap flakes and soap bars; and other readily water-soluble matrices such as salts, sugars, clays, and starches). When extracting delivery particles from finished products which do not disperse readily in water, such as waxes, dryer sheets, dryer bars, and greasy materials, it may be necessary to add detergents, agitation, and/or gently heat the product and diluent in order to release the delivery particles from the matrix. The use of organic solvents or drying out of the delivery particles should be avoided during the extraction steps as these actions may damage the delivery particles during this phase.

For extraction of delivery particles from liquid finished products which are not fabric softeners or fabric enhancers (e.g., liquid laundry detergents, liquid dish washing detergents, liquid hand soaps, lotions, shampoos, conditioners, and hair dyes), mix 20 ml of finished product with 20 ml of DI water. If necessary, NaCl (e.g., 1 to 4 g NaCl) can be added to the diluted suspension in order to increase the density of the solution and facilitate the delivery particles floating to the top layer. If the product has a white color which makes it difficult to distinguish the layers of delivery particles formed during centrifugation, a water-soluble dye can be added to the diluent to provide visual contrast.

The water and product mixture is subjected to sequential rounds of centrifugation, involving removal of the top and bottom layers, re-suspension of those layers in new diluent, followed by further centrifugation, isolation and re-suspension. Each round of centrifugation occurs in tubes of 1.5 to 50 ml in volume, using centrifugal forces of up to 20,000×g, for periods of 5 to 30 minutes. At least six rounds of centrifugation are typically needed to extract and clean sufficient delivery particles for testing. For example, the initial round of centrifugation may be conducted in 50 ml tubes spun at 10,000×g for 30 mins, followed by five more rounds of centrifugation where the material from the top and bottom layers is resuspended separately in fresh diluent in 1.8 ml tubes and spun at 20,000×g for 5 mins per round.

If delivery particles are observed microscopically in both the top and bottom layers, then the delivery particles from these two layers are recombined after the final centrifugation step, to create a single sample containing all the delivery particles extracted from that product. The extracted delivery particles should be analyzed as soon as possible but may be stored as a suspension in DI water for up to 14 days before they are analyzed.

One skilled in the art will recognize that various other protocols may be constructed for the extraction and isolation of delivery particles from finished products and will recognize that such methods require validation via a comparison of the resulting measured values, as measured before and after the delivery particles' addition to and extraction from finished product.

In the following examples, the abbreviations correspond to the materials listed in Table 1.

TABLE 1

| Trade Name | Chemical name | Company/City |
|---|---|---|
| ChitoClear | Chitosan | Primex EHF, Siglufjordur, Iceland |
| Takenate D- | Polyisocyanate | Mitsui Chemicals America, Inc., |
| 110N | prepolymer | Rye Brook, NY |
| | Hydrochloric acid | Avantor Performance Materials, |
| | | LLC, Radnor, PA |
| | Formic acid | Brenntag Great Lakes, LLC, |
| | | Wauwatosa, WI |
| | Potassium | Avantor Performance Materials, |
| | persulfate | LLC, Radnor, PA |
| | Isopropyl myristate | Acme-Hardesty Co., Bule Bell, PA |
| | Hydrogen Peroxide | Avantor Performance Materials, |
| | | LLC, Radnor, PA |

### EXAMPLES

### Comparative Example 1

The comparative example 1 is the same as example 13 in publication US20210252469 A1. A water phase is prepared by dispersing 20.66 g ChitoClear into 439.00 g water while mixing in a jacketed reactor. The pH of the water phase is then adjusted to 4.9 using concentrated HCl under agitation. The water phase temperature is then increased to 85° C. over 60 minutes and then held at 85° C. for a period of time to hydrolyze the ChitoClear. The water phase temperature is then reduced to 25° C. after the hydrolyzing step over a period of 90 minutes. An oil phase is prepared by mixing 159.38 g perfume oil and 23.91 g isopropyl myristate together along with 4.00 g Takenate D-110N at room temperature. The oil phase is added to the water phase under high shear milling to obtain an emulsion. The emulsion is heated to 40° C. over 30 minutes and held for 60 minutes. pH of the emulsion was then adjusted to 2.97 using hydrochloric acid. The emulsion is then heated to 85° C. and maintained at this temperature for 6 hours while mixing. The % degradability is 64.26% at 28 days according to OECD 301B.

### Comparative Example 2

The comparative example 2 is the same as example 10 in publication US20210252469 A1. A water phase is prepared by dispersing 20.66 g ChitoClear into 439.00 g water while mixing in a jacketed reactor. The pH of the water phase is then adjusted to 6.0 using concentrated HCl under agitation. The water phase temperature is then increased to 85° C. over 60 minutes and then held at 85° C. for a period of time to hydrolyze the ChitoClear. The water phase temperature is then reduced to 25° C. after the hydrolyzing step over a period of 90 minutes. An oil phase is prepared by mixing 159.38 g perfume oil and 23.91 g isopropyl myristate together along with 4.00 g Takenate D-110N at room temperature. The oil phase is added to the water phase under high shear milling to obtain an emulsion. The emulsion is heated to 40° C. over 30 minutes and held for 60 minutes. The emulsion is then heated to 85° C. and maintained at this temperature for 6 hours while mixing. Delivery particles are obtained and the % degradability of the delivery particles is 11.07% at 28 days according to OECD 301B.

TABLE 2

| Comparative | | | % degradability |
|---|---|---|---|
| Example | Water phase pH | 1 week leakage | (28 days) |
| 1 | 4.9 | 76.18% | 64.26 |
| 2 | 6.0 | 17.45% | 11.07 |

As can be seen in Table 2, delivery particles obtained at a relatively lower pH (4.9) in Comparative 1, degrade more extensively in the OECD degradability test. However these delivery particles suffer from relatively high leakage. Delivery particles prepared at a slightly higher pH (6) perform better in terms of leakage but suffer from relatively poorer performance in the degradability test. A need exists for delivery particles which have low leakage. Even more desirable are delivery particles which at the same time have relatively high degradability. Achieving a balance of low leakage yet high degradability has been elusive prior to the invention. Even more elusive have been delivery particles having low leakage, high degradability and compatibility with laundry matrices.

### Example 1

An acid and potassium persulfate treated chitosan stock solution is prepared as follows. A potassium persulfate solution was prepared first by dissolving 1.55 g potassium persulfate into 3287.5 g deionized water at 70° C. 154.89 g chitosan ChitoClear was then dispersed into the potassium persulfate solution while mixing in a jacketed reactor. The pH of the chitosan dispersion is then adjusted to 4.30 using 68.37 g concentrated HCl under agitation. The temperature of the chitosan solution is then increased to 85° C. over 60 minutes and then held at 85° C. for a period of time to hydrolyze and depolymerize the chitosan. The temperature is then reduced to 25° C. after the hydrolyzing step over a period of 90 minutes to obtain the acid and potassium persulfate treated chitosan solution. The pH of the chitosan solution is 5.1. The formed chitosan stock solution was used for preparation of capsule in Example 1, 3, 5 and 7.

A water phase is prepared by mixing 420.27 g of the above chitosan stock solution in a jacketed reactor. An oil phase is prepared by mixing 128.30 g perfume and 54.99 g isopropyl myristate together along with 4.01 g Takenate D-110N at room temperature. The oil phase is added to the water phase under high shear milling to obtain an emulsion with desired particle size. The emulsion is heated to 40° C. over 30 minutes and then hold for another 60 minutes. The obtained emulsion is then heated to 90° C. in 60 minutes and maintained at this temperature for 8 hours while mixing before cools down to 25° C. in 90 minutes. The formed capsules have a volume weighted median particle size of 11.71 microns.

### Example 2

An acid and potassium persulfate treated chitosan stock solution is prepared as following. A potassium persulfate solution was prepared first by dissolving 1.55 g potassium persulfate (“KPS”) into 3287.97 g deionized water at 70° C. 154.90 g chitosan ChitoClear was then dispersed into the potassium persulfate solution while mixing in a jacketed reactor. The pH of the chitosan dispersion is then adjusted to 5.10 using 51.72 g concentrated HCl under agitation. The temperature of the chitosan solution is then increased to 85° C. over 60 minutes and then held at 85° C. for a period of time to hydrolyze and depolymerize the chitosan. The temperature is then reduced to 25° C. after the hydrolyzing step over a period of 90 minutes to obtain the acid and potassium persulfate treated chitosan solution. The pH of the chitosan solution is 5.93. The formed chitosan stock solution was used for preparation of capsule in Example 2, 4, 6 and 8.

A water phase is prepared by mixing 422.15 g of the above chitosan stock solution in a jacketed reactor. An oil phase is prepared by mixing 128.30 g perfume and 54.99 g isopropyl myristate together along with 4.01 g Takenate D-110N at room temperature. The oil phase is added to the water phase under high shear milling to obtain an emulsion with desired particle size. The emulsion is heated to 40° C. over 30 minutes and then hold for another 60 minutes. The obtained emulsion is then heated to 90° C. in 60 minutes and maintained at this temperature for 8 hours while mixing before cools down to 25° C. in 90 minutes. The formed capsules have a volume weighted median particle size of 17.64 microns.

TABLE 3

| | | | Median | | | |
|---|---|---|---|---|---|---|
| | % KPS | Water | Particle | 1 wk | | |
| | of | phase | Size | leakage | % Degradability | % Degradability |
| Example # | chitosan | pH | (micron) | (%) | (28 days) | (60 days) |
| 1 | 1 | 5.1 | 11.71 | 83.96 | 27.70 | 34.14 |
| 2 | 1 | 6 | 17.64 | 53.83 | 39.81 | 50.35 |

As can be seen in Table 3, delivery particles based on added persulfate exhibit degradability, but as can be seen in Example 2, with a slight change in pH the leakage also improves relative to Example 1. Moreover Example 2 in addition to improving leakage relative to Example 1, also exhibits degradability of 39.81% in 28 days. This illustrates that persulfate addition enables achieving a surprising balancing of properties by yielding a degradable capsule which also has relatively diminished leakage. Attributes desired in an encapsulate are one or more of low leakage or degradability or compatibility with matrices such as laundry detergent environments. Example 2 illustrates low leakage and degradability. Example 1 illustrates degradability.

### Example 3

A water phase is prepared by mixing 420.27 g of the chitosan stock solution from Example 1 in a jacketed reactor. An oil phase is prepared by mixing 146.63 g perfume and 36.66 g isopropyl myristate together along with 5.55 g Takenate D-110N at room temperature. The oil phase is added to the water phase under high shear milling to obtain an emulsion with desired particle size. The emulsion is heated to 40° C. over 30 minutes and then hold for another 60 minutes. The obtained emulsion is then heated to 90° C. in 60 minutes and maintained at this temperature for 8 hours while mixing before cools down to 25° C. in 90 minutes. The formed capsules have a volume weighted median particle size of 13.32 microns.

### Example 4

A water phase is prepared by mixing 422.15 g of the chitosan stock solution from Example 2 in a jacketed reactor. An oil phase is prepared by mixing 146.63 g perfume and 36.66 g isopropyl myristate together along with 5.55 g Takenate D-110N at room temperature. The oil phase is added to the water phase under high shear milling to obtain an emulsion with desired particle size. The emulsion is heated to 40° C. over 30 minutes and then hold for another 60 minutes. The obtained emulsion is then heated to 90° C. in 60 minutes and maintained at this temperature for 8 hours while mixing before cools down to 25° C. in 90 minutes. The formed capsules have a volume weighted median particle size of 14.29 microns.

TABLE 4

| | | | Median | | | |
|---|---|---|---|---|---|---|
| | % KPS | Water | Particle | 1 wk | | |
| | of | phase | Size | leakage | % Degradability | % Degradability |
| Example # | chitosan | pH | (micron) | (%) | (28 days) | (60 days) |
| 3 | 1 | 5.1 | 13.32 | 44.25 | 13.14 | Not measured |
| 4 | 1 | 6 | 14.29 | 27.20 | 39.97 | 50.52 |

As can be seen in Table 4, delivery particles based on added persulfate exhibit one-week leakage values of 44.25 and 27.20% respectively. Even more surprising, with a subtle adjustment in pH, % degradability increases in these samples from 13.14% to 39.97%. Delivery particles according to the invention consistently display surprising improvement in leakage or degradability or compatibility with matrices. In preferred embodiments, improvement is seen in one category of attributes such as leakage or degradability. More desirably improvement is seen in two categories, such as leakage and degradability, such as shown to be achievable by Example 4 or previously in Example 2. Most desirably improvement is seen in all three categories of leakage, degradability and compatibility. Appropriate selections for example can be drawn from the examples illustrated in Table 8. The parameters of the invention surprising enable assembly of a high performing encapsulate in terms of leakage or degradability or matrix compatibility.

### Example 5

A water phase is prepared by mixing 420.27 g of the chitosan stock solution from Example 1 in a jacketed reactor. An oil phase is prepared by mixing 146.63 g perfume and 36.66 g isopropyl myristate together along with 2.49 g Takenate D-110N at room temperature. The oil phase is added to the water phase under high shear milling to obtain an emulsion with desired particle size. The emulsion is heated to 40° C. over 30 minutes and then hold for another 60 minutes. The obtained emulsion is then heated to 90° C. in 60 minutes and maintained at this temperature for 8 hours while mixing before cools down to 25° C. in 90 minutes. The formed capsules have a volume weighted median particle size of 18.06 microns.

### Example 6

A water phase is prepared by mixing 422.15 g of the chitosan stock solution from Example 2 in a jacketed reactor. An oil phase is prepared by mixing 146.63 g perfume and 36.66 g isopropyl myristate together along with 2.49 g Takenate D-110N at room temperature. The oil phase is added to the water phase under high shear milling to obtain an emulsion with desired particle size. The emulsion is heated to 40° C. over 30 minutes and then hold for another 60 minutes. The obtained emulsion is then heated to 90° C. in 60 minutes and maintained at this temperature for 8 hours while mixing before cools down to 25° C. in 90 minutes. The formed capsules have a volume weighted median particle size of 11.85 microns.

TABLE 5

| | | | Median | | | |
|---|---|---|---|---|---|---|
| | % KPS | Water | Particle | 1 wk | | |
| | of | phase | Size | leakage | % Degradability | % Degradability |
| Example # | chitosan | pH | (micron) | (%) | (28 days) | (60 days) |
| 5 | 1 | 5.1 | 12.71 | 92.82 | 39.16 | 50.51 |
| 6 | 1 | 6 | 11.85 | 67.59 | 49.79 | 55.42 |

Examples 5 and 6 illustrate improved degradability in capsules according to the invention. As pH is adjusted closer to pH 6, a surprising reduction in leakage is noted, in addition to improvement in degradability. These examples reinforce the trend observed in the previous examples that the invention is able to deliver improvements in more than one category of attributes, more particularly in terms of the attributes of leakage, degradability, and compatibility.

### Example 7

A water phase is prepared by mixing 420.27 g of the chitosan stock solution from Example 1 in a jacketed reactor. An oil phase is prepared by mixing 164.96 g perfume and 18.33 g isopropyl myristate together along with 4.01 g Takenate D-110N at room temperature. The oil phase is added to the water phase under high shear milling to obtain an emulsion with desired particle size. The emulsion is heated to 40° C. over 30 minutes and then hold for another 60 minutes. The obtained emulsion is then heated to 90° C. in 60 minutes and maintained at this temperature for 8 hours while mixing before cools down to 25° C. in 90 minutes. The formed capsules have a volume weighted median particle size of 20.54 microns.

### Example 8

A water phase is prepared by mixing 422.15 g of the chitosan stock solution from Example 2 in a jacketed reactor. An oil phase is prepared by mixing 164.96 g perfume and 18.33 g isopropyl myristate together along with 4.01 g Takenate D-110N at room temperature. The oil phase is added to the water phase under high shear milling to obtain an emulsion with desired particle size. The emulsion is heated to 40° C. over 30 minutes and then hold for another 60 minutes. The obtained emulsion is then heated to 90° C. in 60 minutes and maintained at this temperature for 8 hours while mixing before cools down to 25° C. in 90 minutes. The formed capsules have a volume weighted median particle size of 12.56 microns.

TABLE 6

| | | | Median | | | |
|---|---|---|---|---|---|---|
| | % KPS | Water | Particle | 1 wk | | |
| | of | phase | Size | leakage | % Degradability | % Degradability |
| Example # | chitosan | pH | (micron) | (%) | (28 days) | (60 days) |
| 7 | 1 | 5.1 | 12.71 | 92.27 | 22.49 | 27.62 |
| 8 | 1 | 6 | 12.56 | 82.90 | 31.91 | 44.13 |

Examples 7 and 8 illustrate improved degradability in capsules according to the invention. As pH is adjusted closer to pH 6, a reduction in leakage is noted, in addition to improvement in degradability. These examples reinforce the trend observed in the previous examples that the invention is able to deliver improvements in more than one category in terms of the categories of leakage, degradability, and compatibility.

### Example 9

An acid and potassium persulfate treated chitosan stock solution is prepared as follows. A potassium persulfate solution was prepared first by dissolving 1.56 g potassium persulfate into 3303.96 g deionized water at room temperature. 155.68 g chitosan ChitoClear was then dispersed into the potassium persulfate solution while mixing in a jacketed reactor. The pH of the chitosan dispersion is then adjusted to 5.80 using 53.88 g concentrated HCl under agitation. The temperature of the chitosan solution is then increased to 85° C. over 60 minutes and then held at 85° C. for a period of time, such as 2 hours, to hydrolyze and depolymerize the chitosan. The temperature is then reduced to 25° C. after the hydrolyzing step over a period of 90 minutes to obtain the acid and potassium persulfate treated chitosan solution. The pH of the chitosan solution is 5.97.

A water phase is prepared by mixing 2101.81 g of the above chitosan stock solution in a jacketed reactor. An oil phase is prepared by mixing 716.14 g perfume and 179.05 g isopropyl myristate together along with 19.58 g Takenate D-110N at room temperature. The oil phase is added to the water phase under high shear milling to obtain an emulsion with desired particle size. The emulsion is heated to 60° C. over 45 minutes. The emulsion is then heated to 85° C. in 60 minutes and maintained at this temperature for 6 hours while mixing before cools down to 25° C. in 90 minutes. The formed capsules have a volume weighted median particle size of 15.69 microns.

### Example 10

An acid and potassium persulfate treated chitosan stock solution is prepared as follows. A potassium persulfate solution was prepared first by dissolving 1.56 g potassium persulfate into 3303.96 g deionized water at room temperature. 155.68 g chitosan ChitoClear was then dispersed into the potassium persulfate solution while mixing in a jacketed reactor. The pH of the chitosan dispersion is then adjusted to 5.81 using 52.68 g concentrated HCl under agitation. The temperature of the chitosan solution is then increased to 85° C. over 60 minutes and then held at 85° C. for a period of time, such as 2 hours, to hydrolyze and depolymerize the chitosan. The temperature is then reduced to 25° C. after the hydrolyzing step over a period of 90 minutes to obtain the acid and potassium persulfate treated chitosan solution. The pH of the chitosan solution is 5.90.

A water phase is prepared by mixing 2456.58 g of the above chitosan stock solution in a jacketed reactor. An oil phase is prepared by mixing 714.38 g perfume and 178.6 g isopropyl myristate together along with 27.07 g Takenate D-110N at room temperature. The oil phase is added to the water phase under high shear milling to obtain an emulsion with desired particle size. The emulsion is heated to 60° C. over 45 minutes. The emulsion is then heated to 85° C. in 60 minutes and maintained at this temperature for 6 hours while mixing before cools down to 25° C. in 90 minutes. The formed capsules have a volume weighted median particle size of 20.54 microns.

TABLE 7

| | | | Median | | | |
|---|---|---|---|---|---|---|
| | % KPS | Water | Particle | 1 wk | | |
| | of | phase | Size | leakage | % Degradability | % Degradability |
| Example # | chitosan | pH | (micron) | (%) | (28 days) | (60 days) |
| 9 | 1 | 5.97 | 15.69 | 17.60 | 54.64 | 64.82 |
| 10 | 1 | 5.90 | 20.54 | 5.91 | 53.50 | 61.17 |

Examples 9 and 10 illustrate improvements in multiple property categories in terms of improved degradability and improvement in leakage values (lower being better) in capsules according to the invention. As pH is adjusted closer to pH 6, a surprising reduction in leakage is observed, in addition to improvement in degradability. These examples illustrate that the invention is able to deliver improvements in more than one category in terms of the categories of leakage, degradability, and compatibility. Compared to comparative example 1 and 2 it is observed that with redox initiator present (KPS) better performance and degradability is observed.

### Comparative Example 3

A water phase comprising an acid treated chitosan stock solution is prepared as following. 96.24 g chitosan ChitoClear was dispersed into 2044.09 g deionized water at 25° C. while mixing in a jacketed reactor. The pH of the chitosan dispersion is then adjusted to 5.36 using 42.87 g concentrated HCl under agitation. The temperature of the chitosan solution is then increased to 65° C. over 30 minutes, then to 85° C. over 30 minutes, then to 95° C. over 30 minutes, and then held at 95° C. for 2 hours to hydrolyze and depolymerize the chitosan. The temperature is then reduced to 25° C. after the hydrolyzing step over a period of 90 minutes to obtain the acid treated chitosan solution. The pH of the chitosan solution is 5.40.

An oil phase is prepared by mixing 635.63 g perfume and 158.92 g isopropyl myristate together along with 24.06 g Takenate D-110N at room temperature. The oil phase is added to the water phase under high shear milling to obtain an emulsion with desired particle size. The emulsion is heated to 60° C. over 45 minutes, and then to 85° C. in 60 minutes, and then held at 85° C. for 6 hours before cooled down to 25° C. in 90 minutes. The formed capsules have a volume weighted median particle size of 10.06 microns.

### Example 11

A water phase comprising an acid and potassium persulfate treated chitosan stock solution is prepared as following. A potassium persulfate (KPS) solution is prepared by dissolving 0.96 g potassium persulfate into 2056.32 g deionized water at 25° C. while mixing in a jacketed reactor. 96.43 g chitosan ChitoClear was then added into the KPS solution. The pH of the chitosan dispersion is then adjusted to 5.91 using 32.96 g concentrated HCl under agitation. The temperature of the chitosan solution is then increased to 85° C. over 60 minutes, and then held at 85° C. for 2 hours to hydrolyze and depolymerize the chitosan. The temperature is then reduced to 25° C. after the hydrolyzing step over a period of 90 minutes to obtain the acid and potassium persulfate treated chitosan solution. The pH of the chitosan solution is 6.04.

An oil phase is prepared by mixing 636.92 g perfume and 159.24 g isopropyl myristate together along with 24.11 g Takenate D-110N at room temperature. The oil phase is added to the water phase under high shear milling to obtain an emulsion with desired particle size. The emulsion is heated to 60° C. over 45 minutes, and then to 85° C. in 60 minutes, and then held at 85° C. 6 hours, and then cooled down to 25° C. in 90 minutes. The formed capsules have a volume weighted median particle size of 33.97 microns.

### Example 12

An acid and potassium persulfate treated chitosan stock solution is prepared as following. 42.08 g chitosan ChitoClear was dispersed into 893.0 g deionized water at 25° C. while mixing in a jacketed reactor. 0.42 g potassium persulfate added and dissolved. The pH of the chitosan dispersion is then adjusted to 5.87 using 14.40 g concentrated HCl under agitation. The temperature of the chitosan solution is then increased to 65° C. over 30 minutes, then to 85° C. over 30 minutes, then to 95° C. over 30 minutes, and then held at 95° C. for 2 hours to hydrolyze and depolymerize the chitosan. The temperature is then reduced to 25° C. after the hydrolyzing step over a period of 90 minutes to obtain the acid and potassium persulfate treated chitosan solution. The pH of the chitosan solution is 5.90.

A water phase is prepared by mixing 433.6 g of the above chitosan stock solution in a jacketed reactor. An oil phase is prepared by mixing 128.86 g perfume and 32.22 g isopropyl myristate together along with 4.88 g Takenate D-110N at room temperature. The oil phase is added to the water phase under high shear milling to obtain an emulsion with desired particle size. The emulsion is heated to 60° C. over 45 minutes, and then to 95° C. in 60 minutes, and then held at 95° C. for 4 hours, then 1.38 g potassium persulfate added and dissolved, then held at 95° C. for 2 hours, and then cooled down to 25° C. in 90 minutes. The formed capsules have a volume weighted median particle size of 36.25 microns.

### Example 13

An acid and potassium persulfate treated chitosan stock solution is prepared as following. 42.08 g chitosan ChitoClear was dispersed into 893.1 g deionized water at 25° C. while mixing in a jacketed reactor. 4.20 g potassium persulfate added and dissolved. The pH of the chitosan dispersion is then adjusted to 5.94 using 14.35 g concentrated HCl under agitation. The temperature of the chitosan solution is then increased to 65° C. over 30 minutes, then to 85° C. over 30 minutes, and then held at 85° C. for 2 hours to hydrolyze and depolymerize the chitosan. The temperature is then reduced to 25° C. after the hydrolyzing step over a period of 90 minutes to obtain the acid and potassium persulfate treated chitosan solution. The pH of the chitosan solution is 5.36.

A water phase is prepared by mixing 433.6 g of the chitosan stock solution from Example 13 in a jacketed reactor. An oil phase is prepared by mixing 128.86 g perfume and 32.22 g isopropyl myristate together along with 4.88 g Takenate D-110N at room temperature. The oil phase is added to the water phase under high shear milling to obtain an emulsion with desired particle size. The emulsion is heated to 60° C. over 45 minutes, and then to 85° C. in 60 minutes, and then held at 85° C. 6 hours, and then cooled down to 25° C. in 90 minutes. The formed capsules have a volume weighted median particle size of 50.79 microns.

### Example 14

An acid and potassium persulfate treated chitosan stock solution is prepared as following. 42.20 g chitosan ChitoClear was dispersed into 893.1 g deionized water at 25° C. while mixing in a jacketed reactor. 0.42 g potassium persulfate added and dissolved. The pH of the chitosan dispersion is then adjusted to 5.91 using 11.48 g concentrated HCl and 1.25 g 90% Formic Acid under agitation. The temperature of the chitosan solution is then increased to 65° C. over 30 minutes, then to 85° C. over 30 minutes, then to 95° C. over 30 minutes, and then held at 95° C. for 2 hours to hydrolyze and depolymerize the chitosan. The temperature is then reduced to 25° C. after the hydrolyzing step over a period of 90 minutes to obtain the acid and potassium persulfate treated chitosan solution. The pH of the chitosan solution is 5.99. The formed chitosan stock solution was used for preparation of capsules in Examples 14 and 15.

A water phase is prepared by mixing 433.6 g of the chitosan stock solution from Example 14 in a jacketed reactor. An oil phase is prepared by mixing 128.86 g perfume and 32.22 g isopropyl myristate together along with 4.88 g Takenate D-110N at room temperature. The oil phase is added to the water phase under high shear milling to obtain an emulsion with desired particle size. The emulsion is heated to 60° C. over 45 minutes, and then to 95° C. in 60 minutes, and then held at 95° C. 6 hours, and then cooled down to 25° C. in 90 minutes. The formed capsules have a volume weighted median particle size of 33.48 microns.

### Example 15

A water phase is prepared by mixing 433.6 g of the chitosan stock solution from Example 14 in a jacketed reactor. An oil phase is prepared by mixing 128.86 g perfume and 32.22 g isopropyl myristate together along with 4.88 g Takenate D-110N at room temperature. The oil phase is added to the water phase under high shear milling to obtain an emulsion with desired particle size. The emulsion is heated to 60° C. over 45 minutes, and then to 95° C. in 60 minutes, and then held at 95° C. for 4 hours, then 1.38 g potassium persulfate added and dissolved, then held at 95° C. for 2 hours, and then cooled down to 25° C. in 90 minutes. The formed capsules have a volume weighted median particle size of 36.25 microns.

### Example 16

An acid and potassium persulfate treated chitosan stock solution is prepared as following. 42.15 g chitosan ChitoClear was dispersed into 893.1 g deionized water at 25° C. while mixing in a jacketed reactor. 0.42 g potassium persulfate is added and dissolved. The pH of the chitosan dispersion is then adjusted to 5.92 using 8.66 g concentrated HCl and 2.52 g 90% Formic Acid under agitation. The temperature of the chitosan solution is then increased to 65° C. over 30 minutes, then to 85° C. over 30 minutes, then to 95° C. over 30 minutes, and then held at 95° C. for 2 hours to hydrolyze and depolymerize the chitosan. The temperature is then reduced to 25° C. after the hydrolyzing step over a period of 90 minutes to obtain the acid and potassium persulfate treated chitosan solution. The pH of the chitosan solution is 6.01. The formed chitosan stock solution was used for preparation of capsule in Example 16 and 17.

A water phase is prepared by mixing 433.6 g of the chitosan stock solution from Example 16 in a jacketed reactor. An oil phase is prepared by mixing 128.86 g perfume and 32.22 g isopropyl myristate together along with 4.88 g Takenate D-110N at room temperature. The oil phase is added to the water phase under high shear milling to obtain an emulsion with desired particle size. The emulsion is heated to 60° C. over 45 minutes, and then to 95° C. in 60 minutes, and then held at 95° C. for 4 hours, then 1.38 g potassium persulfate added and dissolved, then held at 95° C. for 2 hours, and then cooled down to 25° C. in 90 minutes. The formed capsules have a volume weighted median particle size of 31.68 microns.

### Example 17

A water phase is prepared by mixing 433.6 g of the chitosan stock solution from Example 16 in a jacketed reactor. An oil phase is prepared by mixing 128.86 g perfume and 32.22 g isopropyl myristate together along with 4.88 g Takenate D-110N at room temperature. The oil phase is added to the water phase under high shear milling to obtain an emulsion with desired particle size. The emulsion is heated to 60° C. over 45 minutes, and then to 95° C. in 60 minutes, and then held at 95° C. for 4 hours, then 3.90 g potassium persulfate added and dissolved, then held at 95° C. for 2 hours, and then cooled down to 25° C. in 90 minutes. The formed capsules have a volume weighted median particle size of 31.68 microns.

### Example 18

An acid and potassium persulfate treated chitosan stock solution is prepared as following. 156.60 g chitosan ChitoClear was dispersed into 3321.0 g deionized water at 25° C. while mixing in a jacketed reactor. 1.57 g potassium persulfate added and dissolved. The pH of the chitosan dispersion is then adjusted to 5.93 using 32.05 g concentrated HCl and 9.29 g 90% Formic Acid under agitation. The temperature of the chitosan solution is then increased to 65° C. over 30 minutes, then to 85° C. over 30 minutes, and then held at 85° C. for 2 hours to hydrolyze and depolymerize the chitosan. The temperature is then reduced to 25° C. after the hydrolyzing step over a period of 90 minutes to obtain the acid and potassium persulfate treated chitosan solution. The solution was combined and homogenized with 360 g of stock solution from example 19. The pH of the chitosan solution is 5.99. The formed chitosan stock solution was used for preparation of capsules in Examples 18 and 19.

A water phase is prepared by mixing 433.5 g of the chitosan stock solution from Example 18 in a jacketed reactor. An oil phase is prepared by mixing 128.86 g perfume and 32.22 g isopropyl myristate together along with 4.88 g Takenate D-110N at room temperature. The oil phase is added to the water phase under high shear milling to obtain an emulsion with desired particle size. The emulsion is heated to 60° C. over 45 minutes, then to 85° C. in 60 minutes, then 0.32 g 30% Hydrogen Peroxide (H₂O₂) solution added, and then held at 85° C. for 6 hours, and then cooled down to 25° C. in 90 minutes. The formed capsules have a volume weighted median particle size of 33.89 microns.

### Example 19

A water phase is prepared by mixing 433.5 g of the chitosan stock solution from Example 18 in a jacketed reactor. An oil phase is prepared by mixing 128.86 g perfume and 32.22 g isopropyl myristate together along with 4.88 g Takenate D-110N at room temperature. The oil phase is added to the water phase under high shear milling to obtain an emulsion with desired particle size. The emulsion is heated to 60° C. over 45 minutes, then to 85° C. in 60 minutes, then 0.65 g 30% Hydrogen Peroxide solution added, and then held at 85° C. for 6 hours, and then cooled down to 25° C. in 90 minutes. The formed capsules have a volume weighted median particle size of 30.42 microns.

### Example 20

An acid and potassium persulfate treated chitosan stock solution is prepared as following. 156.55 g chitosan ChitoClear was dispersed into 3320.0 g deionized water at 25° C. while mixing in a jacketed reactor. 1.58 g potassium persulfate added and dissolved. The pH of the chitosan dispersion is then adjusted to 5.95 using 32.05 g concentrated HCl and 9.27 g 90% Formic Acid under agitation. The temperature of the chitosan solution is then increased to 65° C. over 30 minutes, then to 85° C. over 30 minutes, and then held at 85° C. for 2 hours to hydrolyze and depolymerize the chitosan. The temperature is then reduced to 25° C. after the hydrolyzing step over a period of 90 minutes to obtain the acid and potassium persulfate treated chitosan solution. The pH of the chitosan solution is 6.00. The formed chitosan stock solution was used for preparation of capsules in Examples 20 and 21.

A water phase is prepared by mixing 433.5 g of the chitosan stock solution from Example 20 in a jacketed reactor. An oil phase is prepared by mixing 128.86 g perfume and 32.22 g isopropyl myristate together along with 4.88 g Takenate D-110N at room temperature. The oil phase is added to the water phase under high shear milling to obtain an emulsion with desired particle size. The emulsion is heated to 60° C. over 45 minutes, then to 85° C. in 60 minutes, then 1.30 g 30% Hydrogen Peroxide solution added, and then held at 85° C. for 6 hours, and then cooled down to 25° C. in 90 minutes. The formed capsules have a volume weighted median particle size of 25.87 microns.

### Example 21

A water phase is prepared by mixing 433.5 g of the chitosan stock solution from Example 20 in a jacketed reactor. An oil phase is prepared by mixing 128.86 g perfume and 32.22 g isopropyl myristate together along with 4.88 g Takenate D-110N at room temperature. The oil phase is added to the water phase under high shear milling to obtain an emulsion with desired particle size. The emulsion is heated to 60° C. over 45 minutes, then to 85° C. in 60 minutes, then 3.25 g 30% Hydrogen Peroxide solution added, and then held at 85° C. for 6 hours, and then cooled down to 25° C. in 90 minutes. The formed capsules have a volume weighted median particle size of 25.87 microns.

TABLE 8

| | | | | Acid | | | | |
|---|---|---|---|---|---|---|---|---|
| | | % KPS of | % H₂O₂ of | normality | Median | | | % |
| | % KPS of | chitosan | chitosan | ratio | Particle | Free | 1 Week | Aggregates |
| | chitosan | in | in | (HCl/Formic | Size | Oil | Leakage | in SUD |
| | in WP | Emulsion | Emulsion | acid) | (micron) | (%) | (%) | matrix |
| Comparative | 0 | 0 | 0 | 100/0 | 10.06 | 0.2 | 36.72 | 104.25% |
| Example 3 | | | | | | | | |
| Example 11 | 1 | 0 | 0 | 100/0 | 33.97 | 0.19 | 1.54 | 83.66% |
| Example 12 | 1 | 7.07 | 0 | 100/0 | 36.25 | 0.19 | 1.04 | 9.38% |
| Example 13 | 10 | 0 | 0 | 100/0 | 50.78 | 0.09 | 0.80 | 0.39% |
| Example 14 | 1 | 0 | 0 | 80/20 | 33.48 | 0.18 | 1.33 | 107.96% |
| Example 15 | 1 | 7.07 | 0 | 80/20 | 36.25 | 0.18 | 1.20 | 52.07% |
| Example 16 | 1 | 7.07 | 0 | 60/40 | 31.68 | 0.18 | 1.37 | 58.18% |
| Example 17 | 1 | 20 | 0 | 60/40 | 31.68 | 0.20 | 1.30 | 0.12% |
| Example 18 | 1 | 0 | 0.5 | 60/40 | 33.89 | 0.08 | 1.46 | 82.89% |
| Example 19 | 1 | 0 | 1 | 60/40 | 30.42 | 0.1 | 1.92 | 20.29% |
| Example 20 | 1 | 0 | 2 | 60/40 | 25.87 | 0.15 | 2.16 | 1.51% |
| Example 21 | 1 | 0 | 5 | 60/40 | 25.87 | 0.14 | 1.23 | 0.00% |

Examples 11 to 21 illustrate compatibility of delivery particles according to the invention with matrices such as laundry detergent. These are compared to Comparative Example 3. Examples 12 and 17 where redox initiator is added to the water phase and to the emulsion exhibit surprising low leakage, and matrix compatibility attributes. Such delivery particles according to the invention would also exhibit favorable degradability attributes. The table further evidences that % aggregates can be tuned or adjusted by the amount of redox initiator introduced. The attribute of a high level of compatibility is achieved when the redox initiator is added to the water phase and/or the emulsion.

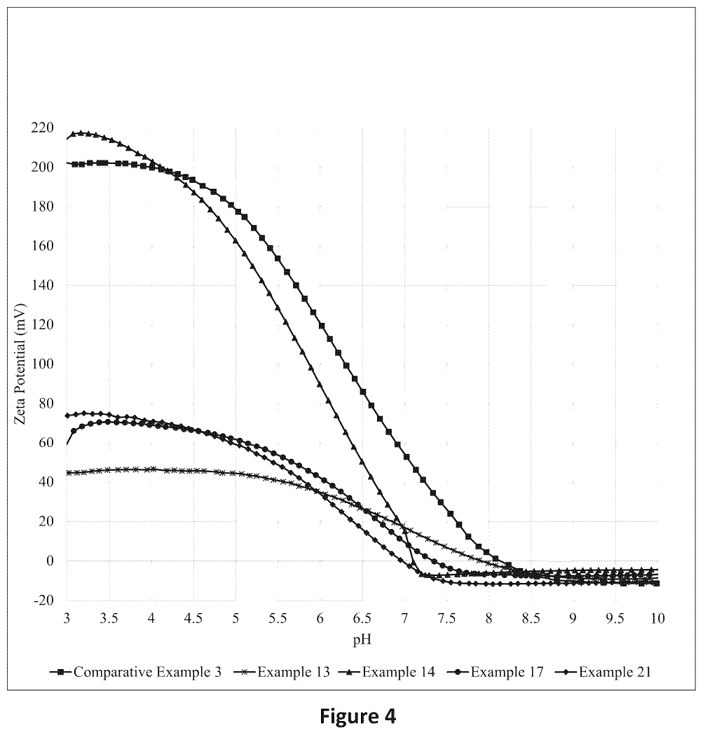

Additionally, FIG. 4 depicts the charge difference of delivery particles made according to various treatments, such as acid treatments and redox initiator addition to the water phase or to the emulsion, as described in the indicated example (i.e., Examples 9, 10, 16, and 18). As the examples show, the steps of the present disclosure enable the zeta potentials to be tailored. For example, the processes of the present disclosure enables lowering or moderating of the zeta potential at pH conditions of use, yielding a more controllable delivery particle, which usefully may be less prone to agglomeration and more compatible with product matrices in end-use applications.

Capsules according to the invention can have core to wall ratios even as high as 95% core to 1% wall by weight. In applications where enhanced degradability is desired, higher core to wall ratios can be used such as 99% core to 1% wall, or even 99.5% to 0.5% by weight or higher.

The shell of the composition in various embodiments according to the invention can be selected to achieve a % degradation target. With appropriate selection of core to wall ratios, the shell of the composition according to the invention can be selected to achieve a % degradation of at least 40% after at least 60 days when tested according to test method OECD 301B.

Uses of singular “a,” “an,” are intended to cover both the singular and the plural, unless otherwise indicated herein or clearly contradicted by context. The terms “comprising,” “having,” “including,” and “containing” are to be construed as open-ended terms. All references, including publications, patent applications, and patents, cited herein are hereby incorporated by reference. Any description of certain embodiments as “preferred” embodiments, and other recitation of embodiments, features, or ranges as being preferred, or suggestion that such are preferred, is not deemed to be limiting. The invention is deemed to encompass embodiments that are presently deemed to be less preferred and that may be described herein as such. All methods described herein can be performed in any suitable order unless otherwise indicated herein or otherwise clearly contradicted by context. The use of any and all examples, or exemplary language (e.g., “such as”) provided herein, is intended to illuminate the invention and does not pose a limitation on the scope of the invention. Any statement herein as to the nature or benefits of the invention or of the preferred embodiments is not intended to be limiting. This invention includes all modifications and equivalents of the subject matter recited herein as permitted by applicable law. Moreover, any combination of the above-described elements in all possible variations thereof is encompassed by the invention unless otherwise indicated herein or otherwise clearly contradicted by context. The description herein of any reference or patent, even if identified as “prior,” is not intended to constitute a concession that such reference or patent is available as prior art against the present invention. No unclaimed language should be deemed to limit the invention in scope. Any statements or suggestions herein that certain features constitute a component of the claimed invention are not intended to be limiting unless reflected in the appended claims.

1. A composition comprising a population of delivery particles,
wherein the delivery particles comprise a core and shell surrounding the core,
wherein the core comprises a benefit agent,
wherein the shell comprises a polymeric material that is the reaction product of a modified chitosan and a cross-linking agent,
wherein the modified chitosan is formed by treating chitosan with a redox initiator,
wherein the redox initiator is selected from the group consisting of a persulfate, a peroxide, and a combination thereof.

2. The composition according to claim 1, wherein the redox initiator is selected from the group consisting of ammonium persulfate, sodium persulfate, potassium persulfate, cesium persulfate, benzoyl peroxide, hydrogen peroxide, and mixtures thereof.

3. The composition according claim 1, wherein the redox initiator and the chitosan are present in a weight ratio of from about 90:10 to about 0.01:99.99, preferably from about 50:50 to about 1:99, more preferably from about 30:70 to about 3:97.

4. The composition according claim 1, wherein the redox initiator is a persulfate, and the shell of the delivery particles comprises sulfur atoms,
preferably wherein the sulfur atoms are present in the shell at a level of from about 0.1% to about 20%, more preferably from about 0.1% to about 10%, even more preferably from about 0.1% to about 1%, by weight of the shell.

5. The composition according to claim 1, wherein the modified chitosan is formed under acidic conditions at a temperature of at least 25° C.,
preferably at pH of 6.5 or less, or even less than pH 6.5, or even at a pH of from 3 to 6.2, or even at pH of from 5 to 6.2.

6. The composition according to claim 1, wherein the modified chitosan is an acid-treated modified chitosan.
wherein the modified chitosan is further treated with a mixture of acids, more preferably a mixture of a first acid and a second acid,
wherein the first acid is a strong acid, and
wherein the second acid is a weak acid,
preferably wherein the first acid and the second acid are present in a normality ratio of from about 20:80 to about 80:20, preferably from about 35:65 to about 65:35.

7. The composition according to claim 6, wherein at least one of the following is true:
(a) the chitosan, prior to treatment with the redox initiator and/or acid, is characterized by a weight average molecular weight of from about 100 kDa to about 600 kDa,
preferably from about 100 kDa to about 500 kDa, more preferably from about 100 kDa to about 400 kDa, more preferably from about 100 kDa to about 300 kDa, even more preferably from about 100 kDa to about 200 kDa;
(b) the modified chitosan is characterized by a weight average molecular weight of from about 1 kDa to about 600 kDa,
preferably from about 5 kDa to about 300 kDa, more preferably from about 10 kDa to about 200 kDa, more preferably from about 15 kDa to about 150 kDa, even more preferably from about 20 kDa to about 100 kDa.

8. The composition according to claim 1, wherein the cross-linking agent comprises a polyisocyanate,
preferably a polyisocyanate selected from the group consisting of a polyisocyanurate of toluene diisocyanate, a trimethylol propane adduct of toluene diisocyanate, a trimethylol propane adduct of xylylene diisocyanate, 2,2′-methylenediphenyl diisocyanate, 4,4′-methylenediphenyl diisocyanate, 2,4′-methylenediphenyl diisocyanate, [diisocyanato(phenyl)methyl]benzene, toluene diisocyanate, tetramethylxylidene diisocyanate, naphthalene-1,5-diisocyanate, 1,4-phenylene diisocyanate, 1,3-diisocyanatobenzene, derivatives thereof, and combinations thereof.

9. The composition according to claim 1, wherein the reaction product is formed in a reaction, wherein the weight ratio of the chitosan present in the reaction to the cross-linker present in the reaction is from about 1:10 to about 1:0.1.

10. The composition according to claim 1, wherein the shell is present in the delivery particles at a level of about 15 wt % or less, by weight of the delivery particles.

11. The composition according to claim 1, wherein the benefit agent is a fragrance material,
preferably a fragrance material comprising perfume raw materials characterized by a log P of from about 2.5 to about 4.5.

12. The composition according to claim 11, wherein the core further comprises a partitioning modifier, optionally present in the core at a level of from about 5% to about 55%, preferably from about 10% to about 50%, more preferably from about 25% to about 50%, by weight of the core,
preferably a partitioning modifier selected from the group consisting of vegetable oil, modified vegetable oil, mono-, di-, and tri-esters of C₄-C₂₄ fatty acids, isopropyl myristate, dodecanophenone, lauryl laurate, methyl behenate, methyl laurate, methyl palmitate, methyl stearate, and mixtures thereof.

13. The composition according to claim 1, wherein the delivery particles are characterized by a volume-weighted median particle size from about 1 to about 100 microns,
preferably from about 10 to about 100 microns, preferably from about 15 to about 50 microns, more preferably from about 20 to about 40 microns, even more preferably from about 25 to about 35 microns.

14. The composition claim 1, wherein the delivery particles are obtainable from a process comprising the steps of:
forming a water phase by treating the chitosan with the redox initiator in the presence of water at a pH of 6.5 or less and at a temperature of at least 25° C., preferably for at least one hour and/or to a time at which the water phase is characterized by a viscosity of less than 1500 cp, preferably less than 500 cp viscosity, to form the modified chitosan,
forming an oil phase, the forming step comprising dissolving together at least one benefit agent and at least one cross-linking agent, preferably a polyisocyanate, optionally with an added oil, preferably a partitioning modifier;
forming an emulsion by mixing the oil phase into an excess of the water phase, preferably under high shear agitation, thereby forming droplets of the oil phase dispersed in the water phase, and optionally adjusting the pH of the emulsion to be in a range from pH 2 to pH 6;
optionally providing a second redox initiator to the emulsion,
wherein the second redox initiator is the same or different as the redox initiator added to the water phase;
curing the emulsion at a temperature of at least 40° C. for a time sufficient to form a shell at an interface of the droplets with the water phase,
the shell comprising the reaction product of the cross-linking agent and the modified chitosan, and
the shell surrounding the core comprising the droplets of the oil phase.

15. The composition claim 1, wherein the delivery particles are obtainable from a process comprising the steps of:
forming a water phase by treating the chitosan in the presence of water at a pH of 6.5 or less and at a temperature of at least 25° C., preferably for at least one hour and/or to a time at which the water phase is characterized by a viscosity of less than 1500 cp, preferably less than 500 cp viscosity,
forming an oil phase, the forming step comprising dissolving together at least one benefit agent and at least one cross-linking agent, preferably a polyisocyanate, optionally with an added oil, preferably a partitioning modifier;
forming an emulsion by mixing the oil phase into an excess of the water phase, preferably under high shear agitation, thereby forming droplets of the oil phase dispersed in the water phase, and optionally adjusting the pH of the emulsion to be in a range from pH 2 to pH 6;
adding a redox initiator to the emulsion to form the modified chitosan,
curing the emulsion at a temperature of at least 40° C. for a time sufficient to form a shell at an interface of the droplets with the water phase,
the shell comprising the reaction product of the cross-linking agent and the modified chitosan, and
the shell surrounding the core comprising the droplets of the oil phase.

16. The composition according to claim 1, wherein the delivery particles are cationic, preferably wherein the delivery particles are characterized by a zeta potential of at least 15 mV at a pH of 4.5.

17. The composition according to claim 1, wherein the modified chitosan is further modified with a modifying compound,
wherein the modifying compound comprises an epoxide, an aldehyde, an α,β-unsaturated compound, or a combination thereof.

18. The composition according to claim 1 wherein the delivery particles have a ratio of core to shell ratio up to 99:1, or even up to 99.5:0.5, on the basis of weight.

19. The composition according to claim 1 wherein the benefit agent is selected from the group consisting of perfume, fragrance, agricultural active, phase change material, essential oil, lubricant, colorant, preservative, antimicrobial active, antifungal active, herbicide, antiviral active, antiseptic active, antioxidant, biological active, deodorant, emollient, humectant, exfoliant, ultraviolet absorbing agent, corrosion inhibitor, silicone oil, wax, bleach particle, fabric conditioner, malodor reducing agent, dye, optical brightener, antiperspirant active and mixture thereof.

20. The composition according to claim 1 wherein the delivery particles have a median particle size of from 1 to 200 microns.

21. The composition according to claim 1, wherein the shells of the delivery particles degrade at least 60% in 60 days when tested according to test method OECD 301B.

22. A process of making a population of delivery particles wherein the delivery particles comprise a core and a shell surrounding the core, the process comprising the steps of:
forming a water phase by treating chitosan with a redox initiator in the presence of water at a pH of 6.5 or less and at a temperature of at least 25° C., preferably for at least one hour and/or to a time at which the water phase is characterized by a viscosity of less than 1500 cp, preferably less than 500 cp viscosity, to form a modified chitosan,
forming an oil phase, the forming step comprising dissolving together at least one benefit agent and at least one cross-linking agent, preferably a polyisocyanate, optionally with an added oil, preferably a partitioning modifier;
forming an emulsion by mixing the oil phase into an excess of the water phase, preferably under high shear agitation, thereby forming droplets of the oil phase dispersed in the water phase, and optionally adjusting the pH of the emulsion to be in a range from pH 2 to pH 6;
optionally providing a second redox initiator to the emulsion to form the modified chitosan
wherein the second redox initiator is the same or different as the redox initiator added to the water phase;
curing the emulsion at a temperature of at least 40° C. for a time sufficient to form a shell at an interface of the droplets with the water phase,
the shell comprising the reaction product of the cross-linking agent and the modified chitosan, and
the shell surrounding the core comprising the droplets of the oil phase.

23. A process of making a population of delivery particles wherein the delivery particles comprise a core and a shell surrounding the core, the process comprising the steps of:
forming a water phase by treating chitosan with an acid in the presence of water at a pH of 6.5 or less and at a temperature of at least 25° C., preferably for at least one hour and/or to a time at which the water phase is characterized by a viscosity of less than 1500 cp, preferably less than 500 cp viscosity,
forming an oil phase, the forming step comprising dissolving together at least one benefit agent and at least one cross-linking agent, preferably a polyisocyanate, optionally with an added oil, preferably a partitioning modifier;
forming an emulsion by mixing the oil phase into an excess of the water phase, preferably under high shear agitation, thereby forming droplets of the oil phase dispersed in the water phase, and optionally adjusting the pH of the emulsion to be in a range from pH 2 to pH 6;
adding a redox initiator to the emulsion to form the modified chitosan,
curing the emulsion at a temperature of at least 40° C. for a time sufficient to form a shell at an interface of the droplets with the water phase,
the shell comprising the reaction product of the cross-linking agent and the modified chitosan, and
the shell surrounding the core comprising the droplets of the oil phase.

24. An article of manufacture incorporating the population of core-shell delivery particles according to claim 1.

25. The article of manufacture according to claim 24 wherein the article is selected from the group consisting of an agricultural formulation, a slurry encapsulating an agricultural active, a population of dry delivery particles encapsulating an agricultural active, an agricultural formulation encapsulating an insecticide, and an agricultural formulation for delivering a preemergent herbicide.

26. The article of manufacture according to claim 25 wherein the agricultural active is selected from the group consisting of an agricultural herbicide, an agricultural pheromone, an agricultural pesticide, an agricultural nutrient, an insect control agent and a plant stimulant.
